(19) United States
(12) Patent Application Publication
(10) Pub. No.: US 2022/0412952 A1
(43) Pub. Date: Dec. 29, 2022

(54) USE OF A CHEMIRESISTOR SENSOR FOR IMPROVING HEALTH

(71) Applicant: NANOSCENT LTD., Mishgav (IL)

(72) Inventors: Orna BARASH, Kiryat Tivon (IL); Yair PASKA, Kiryat Ata (IL); Oren GAVRIELY, Yaad, Misgav (IL); Eran ROM, Yaad, Misgav (IL); Nitzan GIVON, Kfar Blum (IL)

(73) Assignee: NANOSCENT LTD., Mishgav (IL)

(21) Appl. No.: 17/781,436

(22) PCT Filed: Nov. 29, 2020
(86) PCT No.: PCT/IL2020/051232

§ 371 Date: Jun. 1, 2022

Related U.S. Application Data
(60) Provisional application No. 62/942,148, filed on Dec. 1, 2019.

(51) Int. Cl. G01N 33/497; G16H 50/20; G16H 10/40; G16H 20/60

(52) U.S. Cl. CPC G01N 33/497; G16H 50/20; G16H 10/40; G16H 20/60; G01N 2033/4977

(57) ABSTRACT

The present invention provides methods for profiling the microbiome of a subject from a sample obtained from the subject using a Scent Reader/Recorder which detects and records the scent in the headspace of the sample and generates a pattern of sensor signals that can be analyzed using machine learning techniques. The invention further provides methods for detecting changes in the microbiome profile of a subject, and methods for providing health and nutritional recommendations to subjects.

### FIELD OF THE INVENTION

The present invention provides methods for profiling the microbiome of a subject from a sample obtained from the subject using a Scent Reader/Recorder which detects and records the scent in the headspace of the sample and generates a pattern of sensor signals that can be analyzed using machine learning techniques. The invention further provides methods for detecting changes in the microbiome profile of a subject, and methods for providing health and nutritional recommendations to subjects.

### BACKGROUND OF THE INVENTION

The human microbiome (the aggregate of all microbes that reside on or within the human body) is generally not harmful and is even essential for maintaining health. For example, the microbiome is involved in synthesizing various vitamins, breaking down of food, and helping the immune system by assisting in the recognition of dangerous invaders and producing anti-inflammatory compounds.

The microbiome has a baseline balance (homeostasis), which determines human health and sickness. Disruption of this balance may cause susceptibility to different clinical conditions. Early detection of changes in the microbiome may allow intervention, for example by adding probiotics or functional foods to a subject's diet, for returning the microbiome to its baseline.

Advances in DNA sequencing technologies allow comprehensive examination of the microbiome, although these techniques are slow and costly. Thus, there is an unmet need for new methods for analyzing the microbiome in a rapid and inexpensive manner.

Microbes emit volatile by-products to their environment, which are also known as volatile organic compounds (VOCs). Therefore, detection of VOCs in excreted substances such as feces may provide a way to measure the microbiome of subjects, and particularly the gut microbiome. Five hundred compounds identified in feces scent were associated with microbiome metabolism, including methane, aliphatic amines, ammonia, branched chain fatty acids, and derivatives of phenol or indole.

Sensitive, rapid, and relatively inexpensive methods for analyzing the microbiome of subjects would allow consistent and frequent monitoring of the subject's microbiome, which would in turn provide subjects with up-to-date health and nutritional information and recommendations for maintaining or improving microbiome balance and overall health. Such methods are needed in the art.

### SUMMARY OF THE INVENTION

In one embodiment, the present invention provides a method of profiling the microbiome of a subject from a sample obtained from said subject, comprising the steps of: (a) exposing the gaseous phase of said sample to a scent recorder comprising one or more sensors; (b) receiving a pattern of sensor signals from said scent recorder; (c) providing said pattern of sensor signals to a model trained to associate said pattern of sensor signals with a microbiome profile; and (d) determining the microbiome profile of said subject based on said association.

In another embodiment, the present invention provides a method of detecting changes in the microbiome profile of a subject comprising the steps of: (a) profiling the microbiome of said subject based on the gaseous phase of a first sample obtained from said subject at a first timepoint; (b) profiling the microbiome of said subject based on the gaseous phase of a second sample obtained from said subject at a second timepoint; and (c) comparing the microbiome profile of said subject at said first timepoint to the microbiome profile of said subject at said second timepoint, wherein if the microbiome profiles at the two timepoints are different, then a change in the microbiome profile of said subject is detected.

In another embodiment, the present invention provides a method of training a model to associate the microbiome profiles of subjects with patterns of sensor signals from a scent recorder, the method comprising the steps of: (a) providing one or more samples obtained from one of said subjects; (b) exposing the gaseous phase of said one or more samples to a scent recorder; (c) receiving a pattern of sensor signals from said scent recorder; (d) identifying one or more microbes in said sample using molecular techniques; (e) correlating said pattern of sensor signals with said one or more microbes identified; and (f) repeating steps (a) through (e) with one or more samples from one or more additional subjects, to train the model to associate patterns of sensor signals from said scent recorder with said one or more microbes identified.

In another embodiment, the present invention provides a method of recommending/prescribing a diet to a subject comprising the steps of: (a) profiling the microbiome of said subject based on a sample obtained from said subject; (b) diagnosing said subject with a dietary condition based on the microbiome profile; and (c) making a first dietary recommendation to said subject based on said microbiome profile and said dietary condition.

In another embodiment, the present invention provides a method of evaluating the effectiveness of a dietary recommendation in a subject comprising the steps of: (a) recommending/prescribing a diet to a subject; (b) obtaining a second sample from said subject at a second timepoint, wherein said second timepoint is a set time after the implementation of said dietary recommendation by said subject; (c) profiling the microbiome of said subject based on said second sample obtained from said subject; and (d) diagnosing said subject with a dietary condition based on the presence, absence, or relative abundance of at least one microbe in said microbiome profile; wherein (i) if the presence, absence, or relative abundance of said at least one microbe in said microbiome profile of said subject at the second timepoint is at the desired level compared to the level at the first timepoint, then said first dietary recommendation is discontinued and the microbiome of said subject is monitored regularly; (ii) if the presence, absence, or relative abundance of said at least one microbe in said microbiome profile of said subject at the second timepoint is closer to the desired level compared to the level at the first timepoint, then said first dietary recommendation is continued and the microbiome of said subject is monitored regularly until condition (i) is met; and (iii) if the presence, absence, or relative abundance of at least one microbe in said microbiome profile of said subject at the second timepoint is farther from the desired level compared to the level at the first timepoint, then said subject is recommended a second dietary recommendation.

In another embodiment, the present invention provides a method of recommending/prescribing an exercise regimen to a subject comprising the steps of: (a) profiling the microbiome of said subject based on a sample obtained from said subject; (b) evaluating physical fitness parameters of said subject based on the microbiome profile of said subject; and (c) recommending a first exercise regimen to said subject based on said microbiome profile and said physical fitness parameters.

In another embodiment, the present invention provides a method of evaluating the effectiveness of an exercise regimen recommendation in a subject comprising the steps of: (a) recommending an exercise regimen according to claim 10; (b) obtaining a second sample from said subject at a second timepoint, wherein said second timepoint is a set time after the implementation of said exercise regimen by said subject; (c) profiling the microbiome of said subject based on said second sample obtained from said subject; and (d) evaluating physical fitness parameters of said subject based on the microbiome profile of said subject; wherein (i) if the presence, absence, or relative abundance of said at least one microbe in said microbiome profile of said subject at the second timepoint is at the desired level compared to the level at the first timepoint, then said first exercise regimen recommendation is discontinued and the microbiome of said subject is monitored regularly; (ii) if the presence, absence, or relative abundance of said at least one microbe in said microbiome profile of said subject at the second timepoint is closer to the desired level compared to the level at the first timepoint, then said first exercise regimen recommendation is continued and the microbiome of said subject is monitored regularly until condition (i) is met; and (iii) if the presence, absence, or relative abundance of at least one microbe in said microbiome profile of said subject at the second timepoint is farther from the desired level compared to the level at the first timepoint, then said subject is recommended a second exercise regimen recommendation.

In another embodiment, the present invention provides a method of diagnosing a disease, disorder, or condition in a subject comprising the steps of: (a) profiling the microbiome of said subject based on a sample obtained from said subject; and (b) diagnosing said subject with a disease, disorder, or condition based on the microbiome profile of said subject, wherein said disease, disorder, or condition is associated with the microbiome profile of said subject.

In another embodiment, the present invention provides a method of inhibiting or suppressing a disease, disorder, or condition in a subject comprising the steps of: (a) diagnosing said subject with a disease, disorder, or condition on the basis of his/her microbiome profile; (b) selecting a first prophylactic treatment for said subject; and (c) administering said first prophylactic treatment to said subject.

In another embodiment, the present invention provides a method of treating a disease, disorder, or condition in a subject comprising the steps of: (a) diagnosing said subject with a disease, disorder, or condition on the basis of his/her microbiome profile; (b) selecting a first therapeutic treatment for said subject; and (c) administering said treatment to said subject.

### BRIEF DESCRIPTION OF THE DRAWINGS

FIG. 1A. Schematic illustration of a method of evaluating the effectiveness of Daikenchuto (DKT) or other dietary changes using a Scent recorder. A subject in need is administered a dietary supplement or pharmaceutical drug (left panel). The microbiome profile of the subject and changes to the microbiome of the subject are periodically monitored by measuring a sample, such as a stool sample, taken from the subject using a Scent Recorder, such as a NanoScent recorder (middle panel). Finally, the subject is given feedback based on his/her microbiome. For example, based on his/her microbiome profile, the subject may be instructed to continue taking the dietary supplement/pharmaceutical, to increase or decrease the dosage, or to change to a different dietary supplement/pharmaceutical (right panel).

FIG. 1B. Schematic illustration of measurement of fecal samples using NanoScent Scent Recorder. Samples of human excrement (right panel) and mouse fecal pellets (left panel) are placed in a headspace (HS) vial. A NanoScent Scent Recorder which is equipped with sensors configured to detect volatile organic compounds (VOCs) is exposed to the gaseous phase in the HS.

FIG. 2A. Schematic illustration of HeadSpace-Solid-Phase MicroExtraction (HS-SPME) of volatile organic compounds (VOCs) from fecal samples. Fecal samples are placed in a headspace (HS) vial, and VOCs are absorbed from the gas area above the sample (the HS) onto the SPME polymeric fiber. A SPME holder protects the coated fiber and allows positioning of the fiber in the Gas Chromatography (GC) injector port.

FIG. 2B. Schematic illustration of Gas Chromatography (GC) of volatile organic compounds (VOCs). A Solid-Phase MicroExtraction (SPME) holder containing the coated fiber is positioned in the GC injector port, and the absorbed VOCs are desorbed onto the GC column, where the gas sample is separated into its chemical components.

FIG. 2C. Schematic illustration of mass spectrometry (MS) of volatile organic compounds (VOCs). Specific VOCs are identified by MS, thereby providing a full profile of VOCs in a sample.

FIG. 3A. Principal Coordinates Analysis (PCoA) of fecal microbiome of mice administered Daikenchuto (DKT) versus vehicle (saline). 16S ribosomal DNA sequencing was used to detect the microbiome from fecal samples of mice administered saline (blue data points) and mice administered DKT (red data points), and the data is presented in graph form using PCoA. Data points from Day 4 (circles), Day 9 (diamonds), Day 14 (triangles), Day 18 (circle outlines) and Day 22 (stars) are presented. Statistical analysis of the PCoA values demonstrated a significant difference between the group of mice administered DKT and the control group: P-Value=0.036.

FIG. 3B. Alpha diversity metrics of Faith Phylogenetic Diversity (PD) for mice administered Daikenchuto (DKT) versus vehicle (saline). Faith PD measurement of the phylogenetic diversity in the group of mice administered DKT and mice administered saline (control). Faith PD P-Value=0.0043. NC=normal chow; DKT=Daikenchuto; S=saline.

FIG. 3C. Alpha diversity metrics of Pielou Evenness for mice administered Daikenchuto (DKT) versus vehicle (saline). Evenness measurement of the uniformity of the distribution found in the group of mice administered DKT and mice administered saline (control). Evenness P-Value=0.0058. NC=normal chow; DKT=Daikenchuto; S=saline.

FIG. 4A. Class taxa microbiome bar plot of mice administered Daikenchuto (DKT) versus vehicle (saline). Each bar represents the microbiome of the fecal samples collected from a single cage, in which all mice in the cage were administered either DKT (left-side plots) or saline (right-side plots) on days 1-14. Each color represents a different class of micro-organism, as defined in the figure legend. NC=normal chow; DKT=Daikenchuto; S=saline.

FIG. 4B. Genus taxa microbiome bar plot of mice administered Daikenchuto (DKT) versus vehicle (saline). Each bar represents the microbiome of the fecal samples collected from a single cage, in which all mice in the cage were administered either DKT (left-side plots) or saline (right-side plots) on days 1-14. Each color represents a different genus of micro-organism, as defined in the figure legend.

FIG. 4C. Species taxa microbiome bar plot of Human Microbiota Transfer (HMT) mice administered Daikenchuto (DKT) versus vehicle (saline). Each bar represents the microbiome of the fecal samples collected from a single cage, in which all mice in the cage were administered either DKT (left-side plots) or saline (right-side plots) on days 1-14. Each color represents a different species of micro-organism, as defined in the figure legend.

FIG. 5A. Principal component analysis (PCA) of NanoScent Scent Recorder signals for Germ-Free (GF) mice and Human Microbiota Transfer (HMT) mice administered Daikenchuto (DKT) or vehicle (saline). Fecal samples collected from GF mice (orange data points) and HMT mice administered saline (control; blue data points) or DKT (green data points) were analyzed using NanoScent Scent Recorder. The data is presented on a PCA map.

FIG. 5B. Principal component analysis (PCA) of NanoScent Scent Recorder signals for Human Microbiota Transfer (HMT) mice administered Daikenchuto (DKT) or vehicle (saline). Fecal samples collected from HMT mice administered saline (control; orange data points) or DKT (blue data points) were analyzed using NanoScent Scent Recorder, and data was presented on a PCA map. The data from each day of fecal analysis (Days 4, 9, 14, 18 and 22) are circled. The number beside each dot (1-6) represents the cage from which the feces sample was taken.

FIG. 6. Distribution of Volatile Organic Compound (VOC) families in fecal samples from Human Microbiota Transfer (HMT) mice administered Daikenchuto (DKT) or vehicle (saline). Fecal samples collected from HMT mice administered saline (control) or DKT were analyzed using gas chromatography-mass spectrometry (GC-MS) for identification of VOCs. The VOCs were grouped by family (acid, alcohol, ketone, aldehyde, alkene, amine, indole, phenol, silane, thiol, alkane, or furan), with each family represented by a different color.

FIG. 7A. Proposed link between changes in microbiome and Volatile Organic Compounds (VOCs) after Daikenchuto (DKT) treatment: acetic acids. Increased Bacteroides and Bifidobacterium species in the microbiome of DKT-treated mice may increase fecal acetic acid in treated mice, as Bacteroides and Bifidobacterium species, among other bacteria, play a role in the fermentation of undigested carbohydrates leading to the production of acetic acid.

FIG. 7B. Proposed link between changes in microbiome and Volatile Organic Compounds (VOCs) after Daikenchuto (DKT) treatment: indole derivatives. Bacteroides, among other bacteria, play a role in the metabolism of aromatic amino acids, such as tyrosine and tryptophan. Fermentation of those aromatic amino acids produce indole derivatives, such as 3-Ethyl-5-methyl-1H-indole-2-carboxylic acid. Decreased Bacteroides species in the microbiome of DKT-treated mice may decrease production of aromatic amino acids and thereby decrease indole derivative levels (e.g., 3-Ethyl-5-methyl-1H-indole-2-carboxylic acid, right-hand panel) in the gut and feces of DKT-treated mice.

FIG. 7C. Proposed link between changes in microbiome and Volatile Organic Compounds (VOCs) after Daikenchuto (DKT) treatment: oxalic acids. Increased Oxalobacter species in the microbiome of DKT-treated mice may decrease fecal oxalic acid derivatives (e.g., hydrazine, ethyl-ethanedioate, right-hand panel) in treated mice. Oxalic acid is found in a variety of vegetables, fruits, nuts, and beverages, and high levels of excreted oxalic acid is associated with urolithiasis and other conditions. Oxalobacter play a role in the degradation of oxalate which is found in vegetables, fruits, and nuts thereby producing hydrazine, ethyl-ethanedioate.

FIG. 8. Order taxa microbiome bar plot from feces from healthy human subjects. The human microbiome of 11 healthy human subjects was determined using 16S ribosomal DNA sequencing of fecal samples (5 samples for each subject, collected over a period of two weeks). Each bar represents the microbiome of the fecal sample collected from a single sample from a single subject. Each letter (A-K) represents an individual subject.

FIG. 9. Volatile Organic Compound (VOC) families detected by Gas Chromatography-Mass Spectrometry (GC-MS) analysis in human fecal samples. Fecal samples from 11 healthy human subjects were analyzed for VOC content using GC-MS (5 samples for each subject, collected over a period of two weeks). The VOCs were grouped by family (acid, alcohol, aldehyde, alkane, amine, benzene, diazole, ether, furan, indole, ketone, phenol, silane, sulfide, terpene, or urea), with each family represented by a different color.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides methods for analyzing the microbiome of subjects by the scent of a sample from the subject and methods for providing subjects with health and nutritional recommendations, as needed. The methods described herein are sensitive, rapid, relatively inexpensive and allow continuous monitoring of the microbiome.

In some embodiments, the present invention provides methods for profiling the microbiome of a subject from a sample, methods for detecting changes in the microbiome profile of a subject, and methods for providing health and nutritional recommendations using a Scent Reader/Recorder which detects and records the scent in the headspace of the sample and generates a pattern of sensor signals that can be analyzed using, for example, machine learning techniques.

### Microbiome Profiling

In one embodiment, the present invention provides methods of profiling the microbiome of a subject from a sample obtained from the subject. In another embodiment, the present invention provides methods of profiling the microbiome of a subject from a biological sample obtained from the subject. In one embodiment, the method of profiling the microbiome of a subject from a sample comprises the steps of: exposing the gaseous phase of the sample to a Scent Reader/Recorder comprising one or more sensors; receiving a pattern of sensor signals from the scent recorder; providing the pattern of sensor signals to a model trained to associate the pattern of sensor signals with a microbiome profile; and determining the microbiome profile of the subject based on the association.

In one embodiment, the terms “biological sample”, “first biological sample” and “second biological sample” as used herein refer to any biological specimen obtained from a subject. Suitable samples include, without limitation, fecal (stool) sample or body fluid such as blood, blood plasma, serum, urine, vaginal secretion, saliva, sweat, or a combination thereof. In one embodiment, the biological sample comprises a stool sample. In another embodiment, the biological sample comprises a breath sample.

In one embodiment the term “gaseous phase” or “headspace” of a sample, as used herein, refers to any gaseous material above, or surrounding a sample contained in a sampling receptacle/cartridge. In one embodiment, the headspace or gaseous phase of a sample comprises Volatile Organic Compounds (VOCs). As used herein the term “Volatile Organic Compound (VOC)” includes any organic compound that may be evaporated from the subject or originates from the subject, for example, from a biological sample obtained from the subject.

In one embodiment the term “scent”, as used herein, refers to imparting a scent, and can be used interchangeably with aroma, fragrance or odor.

In one embodiment, the term “Scent Reader” or “Scent Recorder”, as used herein, refers to a device configured to detect one or more volatile organic compounds (VOCs) in a sample. In some embodiments, the Scent Reader or Recorder comprises one or more sensors.

In one embodiment, the term “pattern of sensor signals”, as used herein, refers to the reproducible signal profile obtained from the Scent Recorder upon detection of a scent in the headspace of a sample.

In one embodiment, the term “microbiome”, as used herein, refers to the ecological community of microbes, including bacteria, fungi, protozoa and viruses, that live on and inside the human body. In one embodiment, the term “profiling the microbiome” or “microbiome profile” as used herein refers to determining the presence of at least one microbe in a sample from at least one microbiome of a subject. In some embodiments, the microbiome profile comprises the diversity and relative abundance of microbes, specific strains or taxonomic categories, such as species, family, class, etc. A microbiome profile can be determined using any suitable means that can assess, measure or quantify one or more microbes (bacteria, fungi, viruses and archaea) that comprise a microbiome. In some embodiments, a microbiome profile is determined by any one of a number of available molecular techniques, for example, multiplex real-time polymerase chain reaction (PCR), RFLP of the 16S rRNA, amplified rDNA restriction analysis, pyrosequencing, whole genome sequencing, Fluorescent In Situ Hybridization (FISH), shotgun analysis, denaturing or temperature gradient gel electrophoresis (DGGE/TGGE), or other methods known in the art.

In one embodiment, a microbiome profile comprises a single microbiome. In one embodiment, the microbiome comprises an intestinal microbiome, a stomach microbiome, a gut microbiome, an oral microbiome, a skin microbiome, or a combination thereof. In some embodiments, the microbiome comprises the gut microbiome.

In one embodiment, the microbiome profile is determined based on at least one sample from more than one microbiome. For example, a microbiome profile can be determined using at least one sample from a subject's gut microbiome and at least one sample from an oral microbiome.

In one embodiment, a microbiome profile comprises information about the presence or absence or relative abundance of a single microbe. In one embodiment, a microbiome profile comprises information about more than one microbe. In one embodiment, a microbiome profile comprises information about 2 or fewer microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, 12 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, 100 or fewer microbes, 200 or fewer microbes, 300 or fewer microbes, 400 or fewer microbe, 500 or fewer microbes, 600 or fewer microbes, 700 or fewer microbes, 800 or fewer microbes, or 800 or more microbes.

In one embodiment, the terms “determining”, “measuring”, “evaluating”, “assessing”, “assaying”, and “analyzing” can be used interchangeably herein to refer to any form of measurement or assessment and include determining if an element is present or not. These terms can include both quantitative and/or qualitative determinations. Assessing may be relative or absolute. These terms can include use of the machine learning techniques and systems described herein.

In one embodiment, the term “individual”, “subject”, or “patient”, as used herein refers to a human. In some embodiments, the subject may be diagnosed with or suspected of being at high risk for a disease. In some embodiments, the subject is healthy. In one embodiment, the term “healthy”, as used herein refers to a subject in a non-disease state, and/or in state of physical, mental and social well-being.

In one embodiment, in any of the methods as described herein, the subject comprises a human subject. In another embodiment, the subject is a mammal. In another embodiment, the subject is a primate, which in one embodiment, is a non-human primate. In another embodiment, the subject is murine, which in one embodiment is a mouse, and, in another embodiment is a rat. In another embodiment, the subject is canine, feline, bovine, equine, caprine, ovine, porcine, simian, ursine, vulpine, or lupine. In one embodiment, the subject is a chicken or fish. In another embodiment, the subject is anserine, aquiline, assinine, cancrine, cervine, corvine, elapine, elaphine, hircine, leonine, leporine, murine, pavonine, piscine, rusine, or serpentine.

In one embodiment, the subject is an adult. In another embodiment, the subject is a child. In one embodiment, the child is an infant. In one embodiment, the subject is male. In another embodiment, the subject is female.

In another embodiment, the present invention provides methods of detecting changes in the microbiome profile of a subject. In one embodiment the method of detecting changes in the microbiome profile of a subject comprises the steps of: profiling the microbiome of the subject based on the gaseous phase of a first sample obtained from the subject at a first timepoint; profiling the microbiome of the subject based on the gaseous phase of a second sample obtained from the subject at a second timepoint; and comparing the microbiome profile of the subject at the first timepoint to the microbiome profile of the subject at the second timepoint, if the microbiome profiles at the two timepoints are different, then a change in the microbiome profile of the subject is detected.

In one embodiment, term “timepoint” refers to a point in time, or a specific instant. In one embodiment, the terms “first timepoint” and “second timepoint” as used herein refer to different points in time. In some embodiments, the time between a first timepoint and a second timepoint comprises no more than 12 hours. In some embodiments, the time between a first timepoint and a second timepoint comprises no more than 24 hours. In some embodiments, the time between a first timepoint and a second timepoint comprises no more than 48 hours. In some embodiments, the time between a first timepoint and a second timepoint comprises no more than 1 week. In some embodiments, the time between a first timepoint and a second timepoint comprises no more than 2 weeks. In some embodiments, the time between a first timepoint and a second timepoint comprises no more than 4 weeks. In some embodiments, the time between a first timepoint and a second timepoint comprises no more than 1 month. In some embodiments, the time between a first timepoint and a second timepoint comprises no more than 2 months. In some embodiments, the time between a first timepoint and a second timepoint comprises no more than 6 months. In some embodiments, the time between a first timepoint and a second timepoint comprises no more than 12 months. In some embodiments, the time between a first timepoint and a second timepoint comprises no more than 24 months. In some embodiments, the time between a first timepoint and a second timepoint comprises no more than 36 months.

In one embodiment of the methods described herein, the diet of the subject was altered between the first and second timepoints. Such alterations to diet include, but are not limited to, ingestion of one or more food supplements, one or more probiotics, one or more prebiotic foods, one or more functional foods, one or more enriched foods, or a combination thereof.

In one embodiment, the term “functional food” as used herein refers to a food that, besides providing nutrients and energy, has an additional function such as improving health, improving well-being, preventing disease, reducing the risk of disease, or a combination thereof. In some embodiments, the functional food comprises a food that enhances physiological functions, psychological functions, biological activities, or a combination thereof in the subject. In some embodiments, the functional food comprises a food that reduces the risk of a disease, disorder, or condition. In one embodiment, a functional food comprises fruits, vegetables, or other plant sources. In one embodiment, a functional food comprises broccoli, nuts, seeds, grains, or a combination thereof. In another embodiment, a functional food comprises cereals, breads, beverages, or a combination thereof, that are fortified with vitamins, herbs, nutraceuticals, or a combination thereof. In one embodiment, the functional food comprises Daikenchuto (DKT).

In one embodiment, enriched foods comprise foods fortified (enriched) with vitamins and minerals, including, for example, calcium, omega-3, folate and vitamin D.

In one embodiment, food supplements comprise one or more vitamins, minerals, calcium, omega-3, or a combination thereof.

In one embodiment of the methods described herein, the subject was treated for a disease, disorder, or condition between the first and second timepoints. In another embodiment, the changes in the microbiome profile of the subject indicate the development of a disease, disorder, or condition that began or developed between the first and second timepoints in the subject. In one embodiment, the condition comprises pregnancy or ovulation state of the subject.

In one embodiment, the terms “disease”, “disorder”, and “condition” as used herein refer to any interruption, cessation, or disorder of body functions, systems or organs. A subject who is being treated for, is diagnosed with, is susceptible to, or is developing a disease, disorder, or condition may or may not display one or more signs and/or symptoms of the disease, disorder, or condition.

In one embodiment, the disease, disorder, or condition comprises inflammatory bowel disease (IBD), irritable bowel syndrome (IBS), gastrointestinal disorder, colitis, Crohn's disease, diabetes mellitus, cancer, respiratory diseases, metabolic diseases, and neurodegenerative disorders

In another embodiment of the methods described herein, the changes in the microbiome profile of the subject reflect alterations of pharmaceutical intake between the first and second timepoints in the subject. In one embodiment, the term “pharmaceutical intake” as used herein refers to consumption or administration of any pharmaceutical compound, drug, or composition. In one embodiment, the pharmaceutical comprises a medication. In one embodiment, the term “medication” or “medicament” as used herein refers to a substance or agent (e.g., medicine, drug, medicinal application, or remedy) that treats, suppresses, prevents or alleviates the symptoms of disease or illness. A medication may be delivered to a subject by any means, including, without limitation, injection, infusion, oral consumption, inhalation, topical application, or a combination thereof. In one embodiment the medication comprises a prescription medication. In one embodiment, the medication is an over-the-counter medication. In another embodiment, the medication is an antibiotic.

In one embodiment, the pharmaceutical comprises a drug of abuse. In one embodiment, the term “drug of abuse” as used herein refers to any substance (legal or illegal), including medications and pharmaceuticals, that is consumed by a subject and as a result of the consumption, the subject displays addictive behavior comprising craving for the substance, dependency, or a combination thereof. The term also includes combinations of drugs, such as, alcohol and cocaine. The term also includes medicaments/pharmaceuticals abused by a subject who has been prescribed such medication, for example, prescription sleep aids and analgesics. In one embodiment, the drug of abuse comprises a club drug, a stimulant, a depressant, an opioid, a hallucinogen, a psychotropic, an over-the-counter medication, a prescription medication, or a combination thereof. In one embodiment, the depressant comprises alcohol. In one embodiment, the opioid comprises Fentanyl, Hydrocodone, Oxycodone, Oxymorphone, Hydromorphone, Meperidine, Diphenoxylate, Morphine Sulfate, Heroin, or a combination thereof. In one embodiment, the stimulant comprises Cocaine, Synthetic Cathinones (Bath Salts), or a combination thereof. In one embodiment, the psychotropic comprises Kratom. In one embodiment, the club drugs comprise GHB, Rohypnol®, ketamine, MDMA (Ecstasy), Methamphetamine, LSD (Acid), or a combination thereof. In one embodiment, the drug of abuse comprises Marijuana, Synthetic Cannabinoids (K2/Spice), Tobacco/Nicotine, Steroids (Anabolic), or a combination thereof.

In one embodiment of the methods described herein, the level or amount of physical activity of the subject was altered between the first and second timepoints at which the microbiome profile is measured.

In one embodiment, the term “physical activity”, as used herein refers to any bodily movement produced by skeletal muscles that consumes more energy than a resting state, including, without limitation, any sports activities, such as walking, running, exercising, swimming, cycling, and aerobic exercises such as the treadmill, stair climbing machine, etc. In some embodiments, physical activity may be measured by body movement or heart rate.

In one embodiment of the method described herein, the subject was exposed to a different physical environment between the first and second timepoints at which samples were collected for microbiome appraisal. In one embodiment, the term “physical environment”, as used herein refers generally to the site, surroundings or conditions in which a subject lives or resides, which may vary in quality by presence of contaminants/pollutants, which comprise, for example, toxic biological, chemical, physical, or radiological substances, or a combination thereof. All three physical states (solids, liquids, and gases), which encompass air, water, soil, or food, may be included in the elements that make up the environment. In one embodiment, the physical environment of the subject at the first timepoint had a different air quality than the physical environment of the subject at the second timepoint. In one embodiment, the subject was exposed to environmental pollution between the first and second timepoints. In some embodiments, the environmental pollution comprises pesticides, antibiotics, heavy metals, organic pollutants, nanomaterials, or a combination thereof.

In another embodiment, the exposure of the subject to psychological stress or anxiety changed between the first and second timepoints. In one embodiment, the term “psychological stress” as used herein refers to the feeling of strain and/or pressure experienced by a subject surrounding a situation, event, experience, or environmental stimulus. In some embodiments, the psychological stress may cause health issues, which may include susceptibility to physical illnesses such as the common cold, compromised immune system, insomnia, impaired sleeping, development of psychological issues such as depression and anxiety, and higher risks of cardiovascular disease.

### Dietary Recommendations

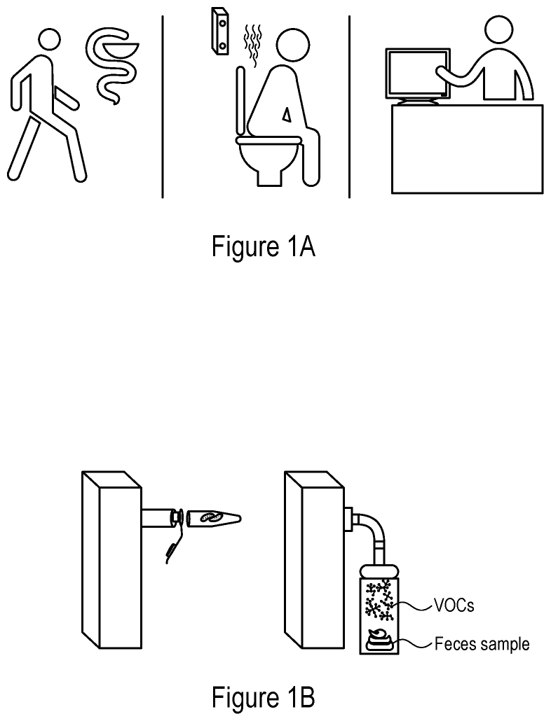

In one embodiment, the present invention provides methods of recommending/prescribing a diet to a subject. Reference is made to FIG. 1A, which is a schematic illustration of a method of evaluating the effectiveness of a dietary change, such as Daikenchuto (DKT) administration. In one embodiment, a subject is administered a dietary supplement or pharmaceutical drug (left panel). The microbiome profile of the subject and changes to the microbiome of the subject are regularly monitored by measuring a sample, such as a stool sample, taken from the subject using a Scent Recorder, such as a NanoScent Scent Reader/Recorder (middle panel). The subject is then given feedback based on his/her microbiome. For example, based on his/her microbiome profile, the subject may be instructed to continue taking the dietary supplement/pharmaceutical, to increase or decrease the dosage, or to change to a different dietary supplement/pharmaceutical (right panel).

In one embodiment, the method of recommending/prescribing a diet to a subject comprises the steps of: profiling the microbiome of the subject based on a sample obtained from the subject; diagnosing the subject with a dietary condition based on the microbiome profile; and making a first dietary recommendation to the subject based on the microbiome profile and the dietary condition. In one embodiment, profiling the microbiome of the subject based on a sample is performed using the methods disclosed herein.

In one embodiment, the term “dietary condition”, as used herein, comprises any nutrient-related disease or condition, which include, without limitation, deficiencies or excesses in the diet, such as deficiencies or excesses in protein levels, essential fatty acids, vitamins, or minerals, obesity and eating disorders, and chronic diseases such as diabetes mellitus.

In one embodiment, the term “dietary recommendation”, “first dietary recommendation”, and “second dietary recommendation”, as used herein comprise enhancing or reducing consumption of fat, oil, or protein-rich foods. In one embodiment, the dietary recommendation comprises ingesting one or more food supplements, one or more probiotics, one or more prebiotic foods, one or more functional foods, one or more enriched foods, or a combination thereof.

In one embodiment, the diet of the subject was altered between the first and second timepoints. Such alterations to diet include, but are not limited to, ingestion of one or more food supplements, one or more probiotics, one or more prebiotic foods, one or more functional foods, one or more enriched foods, or a combination thereof.

In one embodiment, the functional foods comprise food from plant sources, which in one embodiment, comprise fruits, vegetables, or a combination thereof. In one embodiment, the functional food comprises broccoli, nuts, seeds, and grains. In another embodiment, the functional food comprises cereals, breads, and beverages that are fortified with vitamins, herbs, and nutraceuticals or a combination thereof. In one embodiment, the functional food is Daikenchuto (DKT).

In one embodiment, DKT ingestion decreases levels of certain VOCs, which in one embodiment, comprise acetic acid; hydrazine, ethyl-ethanedioate (e.g. oxalic acid derivative); 1h-indole,5-methyl,2-phenyl; and 4-methyl,2-phenyl indole. In one embodiment, the scent reader used in the methods and compositions as described herein detects changes in acetic acid; hydrazine, ethyl-ethanedioate (e.g. oxalic acid derivative); 1h-indole,5-methyl,2-phenyl; and 4-methyl,2-phenyl indole.

In one embodiment, enriched foods comprise foods fortified or enriched with vitamins and minerals, including, for example, calcium, omega-3, folate, vitamin D, or a combination thereof.

In one embodiment, food supplements comprise one or more vitamins, minerals, calcium, omega-3, or a combination thereof.

In one embodiment, the subject has irregular bowel movements. In some of these embodiments, the irregular bowel movements comprise constipation, diarrhea, or a combination thereof.

In one embodiment, the present invention provides methods of evaluating the effectiveness of a dietary recommendation in a subject. In one embodiment the method of evaluating the effectiveness of a dietary recommendation comprises the steps of: recommending/prescribing a diet to a subject; obtaining a second sample from the subject at a second timepoint, where the second timepoint is a set time after the implementation of the dietary recommendation by the subject; profiling the microbiome of the subject based on the second sample obtained from the subject; and diagnosing the subject with a dietary condition based on the presence, absence, or relative abundance of at least one microbe in the microbiome profile; where
i. if the presence, absence, or relative abundance of the at least one microbe in the microbiome profile of the subject at the second timepoint is at the desired level compared to the level at the first timepoint, then the first dietary recommendation is discontinued and the microbiome of the subject is monitored regularly;
ii. if the presence, absence, or relative abundance of the at least one microbe in the microbiome profile of the subject at the second timepoint is closer to the desired level compared to the level at the first timepoint, then the first dietary recommendation is continued and the microbiome of the subject is monitored regularly until condition (i) is met; and
iii. if the presence, absence, or relative abundance of at least one microbe in the microbiome profile of the subject at the second timepoint is farther from the desired level compared to the level at the first timepoint, then the subject is recommended a second dietary recommendation.

In one embodiment, the term “evaluating the effectiveness of a dietary recommendation”, as used herein, includes the use of methods, systems, and computer code, such as machine learning techniques and systems described herein, to determine differences in the presence, absence, or relative abundance of at least one microbe between the microbiome profile at a first timepoint and the microbiome profile at a second timepoint, after the implementation of a dietary recommendation.

### Exercise Recommendations

In one embodiment, the present invention provides methods of recommending/prescribing an exercise regimen to a subject. In one embodiment, the method of recommending/prescribing an exercise regimen to a subject comprises the steps of: profiling the microbiome of the subject based on a sample obtained from the subject; evaluating physical fitness parameters of the subject based on the microbiome profile of the subject; and recommending a first exercise regimen to the subject based on the microbiome profile and the physical fitness parameters. In one embodiment, profiling the microbiome of the subject based on a sample is performed using the methods disclosed herein,

In one embodiment, the term “exercise regimen”, “first exercise regimen” and “second exercise regimen”, as used herein refers to an exercise/workout routine comprising physical activity, as defined herein above, which includes, without limitation, any sports activities, such as walking, running, exercising, swimming, cycling, aerobic exercises such as the treadmill, stair climbing machine, etc. In some embodiments, the exercise regimen comprises aerobic, strength, flexibility, or balance exercises, or a combination thereof. In one embodiment, the term “physical fitness parameters”, as used herein refers to measurable parameters which comprise, without limitation, one or more of cardiovascular/cardiorespiratory endurance (CRE), muscular endurance, stamina, strength, flexibility, body composition, agility, balance, coordination, power, reaction time and speed.

In one embodiment, the present invention provides methods of evaluating the effectiveness of an exercise regimen recommendation in a subject. In one embodiment, the method of evaluating the effectiveness of an exercise regimen recommendation in a subject comprises the steps of: recommending an exercise regimen; obtaining a second sample from the subject at a second timepoint, wherein the second timepoint is a set time after the implementation of the exercise regimen by the subject; profiling the microbiome of the subject based on the second sample obtained from the subject; and evaluating physical fitness parameters of the subject based on the microbiome profile of the subject; where
i. if the presence, absence, or relative abundance of the at least one microbe in the microbiome profile of the subject at the second timepoint is at the desired level compared to the level at the first timepoint, then the first exercise regimen recommendation is discontinued and the microbiome of the subject is monitored regularly;
ii. if the presence, absence, or relative abundance of the at least one microbe in the microbiome profile of the subject at the second timepoint is closer to the desired level compared to the level at the first timepoint, then the first exercise regimen recommendation is continued and the microbiome of the subject is monitored regularly until condition (i) is met; and
iii. if the presence, absence, or relative abundance of at least one microbe in the microbiome profile of the subject at the second timepoint is farther from the desired level compared to the level at the first timepoint, then the subject is recommended a second exercise regimen recommendation.

In one embodiment, the term “evaluating the effectiveness of an exercise regimen recommendation”, as used herein, includes the use of methods, systems, and computer code, such as machine learning techniques and systems described herein, to determine differences in the presence, absence, or relative abundance of at least one microbe between the microbiome profile at a first timepoint and the microbiome profile at a second timepoint, after the implementation of the exercise regimen recommendation.

### Methods of Diagnosis and Treatment

As used herein, the terms “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

In one embodiment, “treating” refers to therapeutic treatment and, in another embodiment, refers to prophylactic or preventative measures. In one embodiment, the goal of treating is to prevent or lessen the disease, disorder, or condition as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof.

In one embodiment, “suppressing” or “inhibiting” refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, the term “diagnosing” as used herein, comprises becoming aware or informing a subject of any medical/health condition. A subject being diagnosed with a disease, disorder, or condition may or may not display one or more signs and/or symptoms of the disease, disorder, or condition.

As used herein, the terms “administering”, “administer”, or “administration” refer to the delivery of one or more pharmaceutical compounds, drugs, or compositions to a subject. In one embodiment, the pharmaceutical compounds, drugs, or compositions are delivered to a subject by any means, including, without limitation, parenteral, enteral, or topical administration. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration. In particular embodiments, an agent or composition is administered parenterally, optionally by intravenous administration or oral administration to a subject.

In one embodiment, the present invention provides methods of diagnosing a disease, disorder, or condition in a subject. In one embodiment, the method of diagnosing a disease, disorder, or condition in a subject comprises the steps of: profiling the microbiome of the subject based on a sample obtained from the subject; and diagnosing the subject with a disease, disorder, or condition based on the microbiome profile of the subject. In one embodiment, the disease, disorder, or condition is associated with the microbiome profile of the subject.

In one embodiment, the present invention provides methods of inhibiting or suppressing a disease, disorder, or condition in a subject. In one embodiment, the method of inhibiting or suppressing a disease, disorder, or condition in a subject comprises the steps of: diagnosing the subject with a disease, disorder, or condition on the basis of his/her microbiome profile; selecting a first prophylactic treatment for the subject; and administering the first prophylactic treatment to the subject.

In one embodiment, the present invention provides methods of treating a disease, disorder, or condition in a subject. In one embodiment, the method of treating a disease, disorder, or condition in a subject comprises the steps of: diagnosing the subject with a disease, disorder, or condition on the basis of his/her microbiome profile; selecting a first therapeutic treatment for the subject; and administering the treatment to the subject.

In one embodiment the method of treating a disease, disorder, or condition in a subject further comprises monitoring the therapeutic effect of the treatment, comprising the steps of: obtaining a second sample from the subject at a second timepoint, where the second timepoint is a set time after the initiation of the treatment; and diagnosing the subject with a disease, disorder, or condition on the basis of his/her microbiome profile, where
i. if the presence, absence, or relative abundance of the at least one microbe in the microbiome profile of the subject at the second timepoint is at the desired level compared to the level at the first timepoint, then the first treatment is discontinued and the microbiome of the subject is monitored regularly;
ii. if the presence, absence, or relative abundance of the at least one microbe in the microbiome profile of the subject at the second timepoint is closer to the desired level compared to the level at the first timepoint, then the first treatment is continued and the microbiome of the subject is monitored regularly until condition (i) is met; and
iii. if the presence, absence, or relative abundance of at least one microbe in the microbiome profile of the subject at the second timepoint is farther from the desired level compared to the level at the first timepoint, then the subject is treated with a second treatment.

In one embodiment, the term “monitoring the therapeutic effect of the treatment”, as used herein, includes the use of methods, systems, and computer code, such as the machine learning techniques and systems described herein, to determine differences in the presence, absence, or relative abundance of at least one microbe between the microbiome profile at a first timepoint and the microbiome profile at a second timepoint, after the implementation of first treatment.

In one embodiment, the microbiome comprises low levels of Faecalibacterium prausnitzii. In one embodiment, the microbiome comprises high levels of Prevotella copri, Bacteroides vulgates, or a combination thereof. In one embodiment, the microbiome comprises low levels of Lactobacillus species. In one embodiment, the microbiome comprises a low diversity of microbial organisms.

In one embodiment, the disease, disorder, or condition comprises inflammatory bowel disease (IBD). In one embodiment, the disease, disorder, or condition comprises obesity, type 2 diabetes-insulin resistant, or a combination thereof. In one embodiment, the disease, disorder, or condition comprises lactose intolerance. In one embodiment, the disease, disorder, or condition comprises allergic asthma. In one embodiment, the subject was exposed to antibiotics during the perinatal or neonatal period.

In one embodiment, the disease, disorder, or condition comprises a gastrointestinal disorder, obesity, or diabetes mellitus. In one embodiment, the gastrointestinal disorder comprises inflammatory bowel disease (IBD). In some embodiments, IBD comprises colitis or Crohn's disease. In one embodiment, colitis comprises infectious colitis, ulcerative colitis, ischemic colitis, microscopic colitis, lymphocytic colitis, collagenous colitis, diversion colitis, chemical colitis, chemotherapy-induced colitis or radiation colitis.

In one embodiment, the gastrointestinal disorder comprises irritable bowel syndrome (IBS) or irregular bowel movements. In one embodiment, the irregular bowel movements comprise constipation, diarrhea, or a combination thereof.

In one embodiment, the disease, disorder, or condition does not comprise diabetes, cystitis, dehydration, vitamin deficiency, influenza (flu), unbalanced diet, constipation, diarrhea, internal ingestion of foreign objects, or a combination thereof. In one embodiment, the disease, disorder, or condition excludes diabetes, cystitis, dehydration, vitamin deficiency, influenza (flu), unbalanced diet, constipation, diarrhea, internal ingestion of foreign objects, or a combination thereof.

### Scent Reader/Recorder

Some embodiments of the methods of the present invention include profiling of the microbiome of a subject from a sample, using a Scent Reader/Recorder which detects the scent in the headspace of samples and generates a pattern of sensor signals.

In another embodiment, the Scent Recorder comprises one or more sensors configured to detect the microbiome profile of a subject from a sample obtained from the subject. In one embodiment, the microbiome profile is detected indirectly from the sample. In another embodiment, the microbiome profile is detected directly from the sample.

In another embodiment, the Scent Recorder comprises one or more sensors configured to detect one or more volatile organic compounds (VOCs) from a sample obtained from a subject.

In one embodiment, a VOC as described herein comprises 1-methyl 4-methyl (1-methylethyl) Benzene, 2-methyl butanal, 3-methyl 1-indole, 3-Methyl phenol, 4-methyl Phenol, Benzene ethanol, Dihydroxy 2,5,8,11,14, Dimethyl disulfide, Dimethyl trisulfide, acetic acid, Propanoic Acid, Styrene, α-pinene, β-caryophyllene, γ-terpinene, δ-carene, hydrazine, ethyl-ethanedioate (e.g. oxalic acid derivative); 1h-indole,5-methyl,2-phenyl; 4-methyl,2-phenyl indole, or a combination thereof. In one embodiment, the SCENT RECORDER detects changes in the levels of acetic acid; hydrazine, ethyl-ethanedioate (e.g. oxalic acid derivative); 1h-indole,5-methyl,2-phenyl; 4-methyl,2-phenyl indole, or a combination thereof.

In one embodiment, the one or more sensors comprise one or more chemi-resistors. In some embodiments, the one or more chemi-resistors comprise metallic nanoparticles coated with an organic ligand shell, metal oxide sensor (MOS), a catalytic near infrared (IR) sensor, a photoionization detector (PID), an IR open path sensor, a portable gas-chromatography mass spectrometer (GC-MS), or electro-chemical sensor.

In one embodiment, the SCENT RECORDER further comprises one or more of:
a. a chamber for holding the one or more sensors;
b. a gas circulation system for directing VOCs in a gas phase towards the one or more sensors;
c. a regeneration device for regenerating the one or more sensors;
d. a holder for holding an absorbing material comprising the VOCs collected from the sample.

In one embodiment, the gas circulation system comprises a fan, a pump, one or more gas monitoring sensors, one or more valves, or a combination thereof.

In one embodiment, the regeneration device comprises a heating element, a vacuum pump, a stream of gas, or a combination thereof.

In one embodiment, the absorbing material is configured to absorb VOCs from the sample. In one embodiment, the VOCs comprise 1-methyl 4-methyl (1-methylethyl) Benzene, 2-methyl butanal, 3-methyl 1-indole, 3-Methyl phenol, 4-methyl Phenol, Benzene ethanol, Dihydroxy 2,5,8,11,14, Dimethyl disulfide, Dimethyl trisulfide, acetic acid, Propanoic Acid, Styrene, α-pinene, β-caryophyllene, γ-terpinene, δ-carene, hydrazine, ethyl-ethanedioate (e.g. oxalic acid derivative); 1h-indole,5-methyl,2-phenyl; and 4-methyl,2-phenyl indole or a combination thereof.

In one embodiment, the SCENT RECORDER comprises a NanoScent®-Scent Reader/Recorder. Different versions of NanoScent Scent Recorders that are suitable for performing the methods described herein are described, inter alia, in International Patent Publication No. WO 2019/135232 and International Application No. PCT/IL2019/050591, which are incorporated herein by reference in their entirety.

In one embodiment, the chemi-resistors comprise a chemi-resistor sensor comprising:
a. two electrodes;
b. a sensing element
   i. electrically connected to the two electrodes;
   ii. comprising a nanoparticle core made from a conductive material comprising Ir, Ir-alloy, IrOx, Ru, Ru-alloy, RuOx, or any combination thereof, and
   iii. having an average diameter of 100 nm at most; and
c. a plurality of organic ligands bonded on one side of the nanoparticle core and capable of interacting with volatile organic compounds (VOCs) from the gaseous phase of the sample.

In one embodiment, the nanoparticle core is at least partially covered with an oxide layer comprising at least one of: IrOx and RuOx.

In one embodiment, the nanoparticle core has a crystalline structure. In another embodiment, the nanoparticle core comprises an amorphous structure.

In one embodiment, the nanoparticle core comprises a mixed structure having a first material coated by a second material. In one embodiment, the first material comprises Jr, Jr-alloy, Ru, Ru-alloy, or a combination thereof. In one embodiment, the second material comprises IrOₓ, RuOₓ, or a combination thereof.

In one embodiment, the plurality of organic ligands comprises amine like dodecylamine, diazoniums, silanes, carboxylic acids, tri-chloro, methoxy, ethoxy, tri hydroxide, di-chloro, chloro, or a combination thereof.

### Trained Model

Some embodiments of the present invention provide methods of profiling the microbiome of a subject from a sample, using a Scent Reader/Recorder which detects the scent in the headspace of samples and generates a pattern of sensor signals that can be analyzed using machine learning techniques, using a trained model.

In one embodiment, the trained model may be trained using a training set of data consisting of patterns of sensor signals generated by the NS which has been provided with samples obtained from several subjects. For training the model, the training set of data may, in one embodiment, be obtained from samples having known microbiome profiles.

In one embodiment the present invention provides a system for training a model to profile the microbiome of a subject. In one embodiment, the system comprises: a scent recorder comprising one or more sensors configured to detect one or more volatile organic compounds (VOCs) from a sample obtained from a subject. In another embodiment, the system comprises a memory storage unit. In another embodiment, the system comprises a controller, which, in one embodiment, is configured to:
i. correlate the pattern of sensor signals from the scent recorder when exposed to a sample with the one or more microbes identified using molecular techniques in the sample;
ii. repeat step (i) with a different sample, to train the model; and
iii. store the trained model in the memory storage unit.

In one embodiment, the molecular techniques for identifying one or more microbes include, without limitation, multiplex real-time polymerase chain reaction (PCR), RFLP of the 16S rRNA, amplified rDNA restriction analysis (ARDRA), pyrosequencing, whole genome sequencing, Fluorescent In Situ Hybridization (FISH), shotgun analysis, denaturing or temperature gradient gel electrophoresis (DGGE/TGGE), or a combination thereof.

In one embodiment, the memory unit comprises a hard disk drive, a universal serial bus (USB) device or a cloud based storing service in communication with the system.

In one embodiment, the sample comprises a stool sample, a breath sample, or a saliva sample.

In one embodiment the present invention provides a method of training a model to associate the microbiome profiles of subjects with patterns of sensor signals from a scent recorder. In one embodiment, the method of training a model to associate the microbiome profiles of subjects with patterns of sensor signals from a scent recorder comprises the steps of:
a. providing one or more samples obtained from one of the subjects;
b. exposing the gaseous phase of the one or more samples to a scent recorder;
c. receiving a pattern of sensor signals from the scent recorder;
d. identifying one or more microbes in the sample using molecular techniques;
e. correlating the pattern of sensor signals with the one or more microbes identified; and
f. repeating steps (a) through (e) with one or more samples from one or more additional subjects, to train the model to associate patterns of sensor signals from the scent recorder with the one or more microbes identified.

In one embodiment, the molecular techniques for identifying one or more microbes in a sample include, among others, multiplex real-time polymerase chain reaction (PCR), RFLP of the 16S rRNA, amplified rDNA restriction analysis (ARDRA), pyrosequencing, whole genome sequencing, Fluorescent In Situ Hybridization (FISH), shotgun analysis, denaturing or temperature gradient gel electrophoresis (DGGE/TGGE), or a combination thereof.

### Kits

The present invention further provides kits for sample collection and analysis. In one embodiment, the kit comprises: one or more means for collecting a sample obtained from a subject; and instructions of use.

In one embodiment, the kit further comprises a scent recorder having one or more sensors configured to detect volatile organic compounds (VOCs). In one embodiment, the sensors comprise one or more chemi-resistors. In some embodiments the chemi-resistors comprise metallic nanoparticles coated with an organic ligand shell, metal oxide sensor (MOS), a catalytic near infrared (IR) sensor, a photoionization detector (PID), an IR open path sensor, a portable gas-chromatography mass spectrometer (GC-MS), or electro-chemical sensor.

In one embodiment, the scent recorder further comprises a chamber for holding the one or more sensors. In another embodiment, the scent recorder further comprises a gas circulation system for directing VOCs in a gas phase towards the one or more sensors. In another embodiment, the scent recorder further comprises a regeneration device for regenerating the one or more sensors. In another embodiment, the scent recorder further comprises a holder for holding an absorbing material comprising the VOCs collected from the sample. In another embodiment, the scent recorder further comprises any combination of the above.

In one embodiment, the gas circulation system comprises a fan, a pump, one or more gas monitoring sensors, one or more valves, or a combination thereof.

In one embodiment, the regeneration device comprises a heating element, a vacuum pump, a stream of gas, or a combination thereof.

In one embodiment, the absorbing material is configured to absorb one or more VOCs from the sample.

In one embodiment, the sample comprises a biological sample. In one embodiment, the biological sample comprises a stool sample. In some embodiments, the means for collecting the sample comprises a sample collection paper. In one embodiment, the means for collecting the sample comprises a sampling receptacle with screw cap. In another embodiment, the means for collecting the sample comprises disposable gloves. In another embodiment, the means for collecting the sample comprises a collection spoon. In another embodiment, the means for collecting the sample comprises a combination of the above.

In another embodiment, the biological sample comprises a breath sample. In some of these embodiments, the means for collecting the biological sample comprises a breath sampler. In another embodiment, the means for collecting the biological sample comprises a breath sample cartridge. In another embodiment, the means for collecting the biological sample comprises a combination of the above.

In another embodiment, the biological sample comprises a saliva sample, a urine sample, a sweat sample, a blood sample, a vaginal secretion, or a combination thereof.

In one embodiment, the kit comprises instructions of use. In one embodiment, the kit comprises instructions for performing the methods herein described. In one embodiment, the kit comprises instructions for collecting a sample. The instructions may be printed directly on the container (when present), or as a label applied to the container, or as a separate sheet, pamphlet, card, or folder supplied in or with the container.

### Definitions

Unless specifically stated otherwise herein, references made in the singular may also include the plural. For example, “a” and “an” may refer to either one, or one or more.

The definitions set forth herein take precedence over definitions set forth in any patent, patent application, and/or patent application publication incorporated herein by reference.

Listed herein are definitions of various terms used to describe the present invention. These definitions apply to the terms as they are used throughout the specification (unless they are otherwise limited in specific instances) either individually or as part of a larger group.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe addition more embodiments. It is also to be understood that each individual element of the embodiments is meant to be combined with any and all other elements from any embodiment to describe an additional embodiment.

### EXAMPLES

### Example 1

### Microbiome Analysis by 16S Ribosomal DNA Sequencing

DNA extraction and amplification: microbial genomic DNA was extracted from fecal samples using beadbeating. For each sample, bacterial 16S rRNA gene sequences were amplified by PCR using barcoded universal primers 515F and 806R, containing Illumina adapter sequences which target the highly conserved V4 region. Equimolar ratios of amplicons from individual samples were pooled prior to sequencing on the Illumina platform using high throughput screening (Illumina MiSeq instrument). Sequences were analyzed using QIIME and taxonomy was assigned using the Greengenes database. Both α diversity (within sample) and β diversity (between samples) were calculated.

In silico Metagenomics: Predictions regarding the functional composition of the microbiome were made on 16S rRNA-derived features using PICRUSt. Using KEGG pathway metadata, KEGG orthologs were categorized by their function to level 3 of the pathway hierarchy. Group differences in functional diversity were calculated using the Shannon Index and analyzed using the Mann Whitney U test. PCoA plots were created using Bray-Curtis distances with the QIIME v1.8.0 software suite, and differences between the functional profiles of control and study groups were analyzed using the analysis of similarities (ANOSIM) test. Differential functional abundance of categorized gene counts was analyzed using the G-test of goodness-of-fit and the Benjamini Hochberg correction for multiple comparisons. Data Analyses were performed using QIIME and the R statistical software. Non-normal microbiome frequencies and relative proportion data were transformed, if required, to fit the assumptions of the statistical models. The combinations related to study arm and disease severity were tested using supervised methods, (e.g. developing linear classifiers for the separation of responders vs. non responders and testing the positive/negative contribution of each feature to the classification) and projections into dimension best separating the sets using either SPCA (Supervised Principle Component Analysis) or LDA (Linear Discriminant Analysis).

### Example 2

### Detecting Volatile Organic Compounds (VOCs) by Solid-Phase Microextraction (SPME) Combined with Gas Chromatography-Mass Spectrometry (GC-MS)

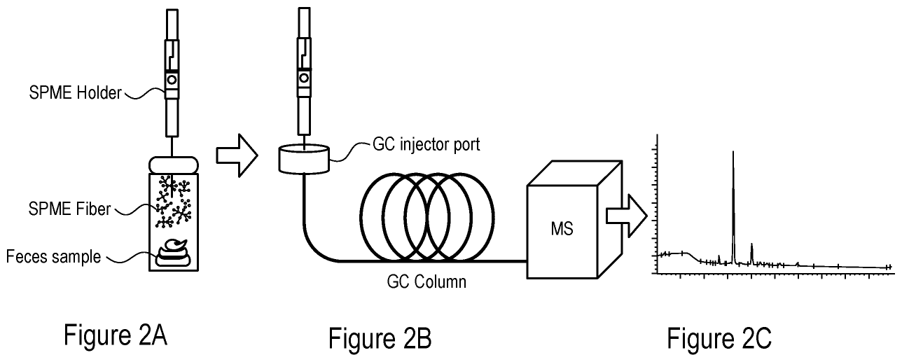

Volatile organic compounds (VOCs) in fecal samples were analysed by gas chromatography-mass spectrometry (GC-MS). A schematic illustration of the process is depicted in FIGS. 2A-C. First, the VOCs are extracted from the feces samples by HeadSpace-Solid-Phase MicroExtraction (HS-SPME) (FIG. 2A). This method used a polymeric fiber (e.g. SPME) to absorb VOCs from the gas area above the sample (i.e. HS) (FIG. 2A). The absorbed VOCs are later desorbed, using an oven, onto the GC column, for their separation (FIG. 2B). The temperature of the oven which ranges between 5° C. to 400° C., is one of the main characteristics that distinguishes one program from another. Here, the feces samples were analysed for VOCs using the following program: 4° C. for 1 min; heat to 220° C. for 4 min at a rate of 5° C./min; and heat to 240° C. for 10 min at a rate of 5° C./min. Specific VOC's are identified by their mass spectrum (FIG. 2C).

In the following examples, either 10 pellets of mice feces or 0.5 gr of human excrement were placed in a 10 mL headspace (HS) vial with 3 ppm of 4-methyl, 2-pentanol, as an internal standard. Vials were placed on automatic HS coupled to GC-MS. Vials were left for 30 min at 60° C. to reach equilibrium of the fecal matter with the headspace. Subsequently, a SPME covered with grey fiber (containing Divinylbenzene/Carboxen/Polydimethylsiloxane) was inserted into the vial for headspace absorption for 3 minutes. VOCs were separated by gas chromatography and identified by mass spectrometry, as described above.

### Statistical Methods

The dataset obtained from the GC-MS was analysed using Wilcoxon-Kruskal statistical test. This statistical test identifies the VOCs that distinguish between samples having different parameters (e.g. diet, age, gender, etc.).

### Example 3

### Scent Analysis Using a NanoScent-Scent Recorder

2-3 pellets of mice feces (as depicted in the left panel of FIG. 1B) or samples of human excrement (depicted in the right panel of FIG. 1B) were analysed by the NanoScent®-Scent Recorder. Different versions of NanoScent-Scent Recorders that are suitable for performing the methods described herein are described, inter alia, in International Patent Publication No. WO 2019/135232 and International Application No. PCT/IL2019/050591, which are incorporated herein by reference in their entirety.

The data collected from the NanoScent Scent Recorder device includes two sets of columns: metadata columns and data columns. The metadata columns include generation time, sample ID, title, and additional experiment specific metadata such as age and gender in the case of the human study experiment. The data columns are features that are extracted from the resistivity readings of each sensor. The device contains 8 sensors, and therefore there are 5 groups of feature columns prefixed GNP1-GNP8. Within each group are the following parameters:
avg. The average of the measured resistivity samples
dr. The delta in the response, max-min
dr/rmin. The delta divided by the minimal resistivity
dr/rmax. The delta divided by the maximal resistivity
exp_a, exp_b, exp_c. The resistors response behaves as exponential deterioration (exponential decay). Therefore, it is approximated as: exp_a+exp_b*e\hat{ }(exp_c).
   The feature extraction phase results in a 40-dimensional vector for each sample.
   In one embodiment, the scent analysis via NanoScent Scent Recorder enables determining:
1. Which VOCs are dominant for each parameter and which VOCs can be used to separate between two groups (e.g. people who practice sports vs. people who do not); and
2. Which subset of features from the NanoScent Scent Recorder's 8 sensors is dominant in the separation between the parameters.
   The VOCs and sensors that perform the separation may then be correlated.

### Example 4

### Machine Learning Algorithm

Goal: To obtain a trained model using machine learning techniques that can infer different parameters (e.g. diet, age, gender, etc.) of a subject from an unknown sample.

Method: Examples 1-3 result in 3 datasets: DNA sequencing of the gut microbiome; GC-MS dataset; and NanoScent Scent Recorder dataset. Supervised machine learning algorithms were applied to all three datasets to obtain trained models.

Supervised learning is a machine learning technique where the machine learns to map inputs to outputs based on a training set. The training set consists of input-output pairs. A supervised learning algorithm analyses the training set and produces a trained model. When presented with an unknown input the trained model infers the output.

Three different algorithms of supervised machine learning were used: Random forest, Support vector machines and Neural networks. For example, for scent analysis, scents of several flowers are measured using a scent reader/recorder as the input, the name of the flower is provided as the output. After taking several measurements for each flower under various conditions, 70% of the input-output pairs are used to train the 3 different algorithms (training set). The remaining 30% of the measurements are then used to test the models (test set). Each input from the test set is presented to each algorithm to yield 3 answers: one from each algorithm. The majority of answers is taken as the final output. In case the 3 algorithms gave 3 different answers the output is chosen arbitrarily. The test set output is then compared with the known output for calculating the accuracy of the trained model.

### Example 5

### DKT Administration to Mice Alters Gut Microbiome, VOCs, and Fecal Scent

Goal: To evaluate the effect of Daikenchuto (DKT) administration on feces scent, the gut microbiome composition and VOCs. Specific goals were:
Detecting differences among odours of mice feces (odour refers to the total pattern of VOCs, which influence feces scent).
Detecting changes in microbiome and fecal scent after functional food (i.e. Daikenchuto (DKT)) administration
Correlating mice gut microbiome and fecal scent

Treatment Method—Mice: 15 Germ Free (GF) mice were divided into two groups of 7-8 mice each. The two groups were further divided into three cages (e.g. 3 mice in each cage). Human feces were implanted into the gut of all GF mice (i.e. Human Microbiota Transfer—HMT) for establishing a human microbiome in the GF mice (Day 1). The fecal sample for transplantation was provided by a healthy, female volunteer (36Y) who also provided comprehensive clinical information. On the following day, DKT was administered at a dose of 1200 mg/kg to the test group through oral gavage, while the mice in the control group were orally administered saline in the same manner. DKT and saline were administered every day for 14 days (Days 2-15, relative to the day of HMT). All mice were weighed throughout the experiment to ensure weight gain.

Fresh fecal samples for analysis were collected from all cages on days 4, 9, 14, 18, and 22, relative to the day of HMT (i.e. Day 9 refers to nine days after HMT). Day 15 is the last day of DKT administration, and Day 22 is one week after the last DKT administration.

Fresh feces were collected from the cages and transferred to −20° C. On each collection day, 13 pellets were collected from each cage; of these, 10 pellets were collected for GC-MS analysis, one pellet was collected for DNA sequencing, and two pellets were exposed to the NanoScent Scent Reader.

Results:

### Microbiome Analysis by 16S Ribosomal DNA Sequencing:

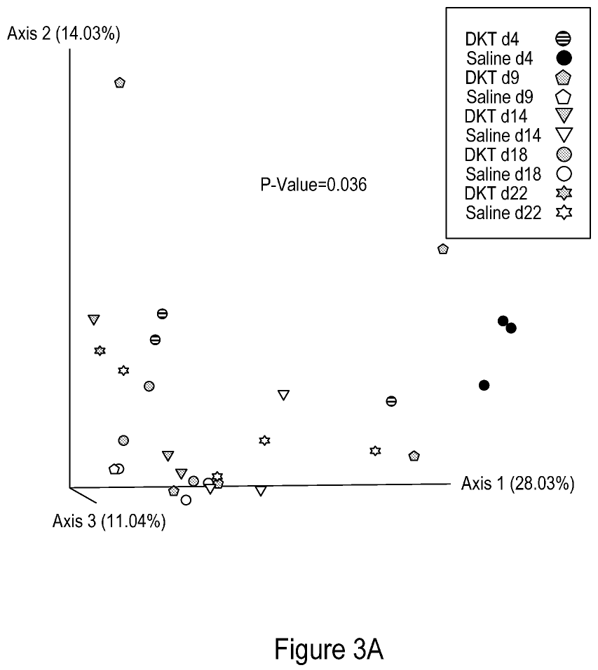

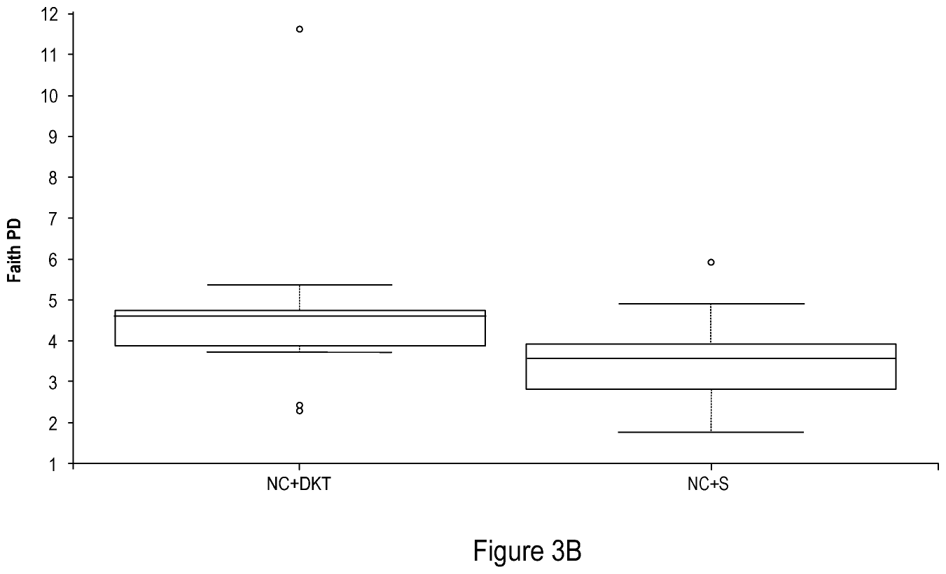

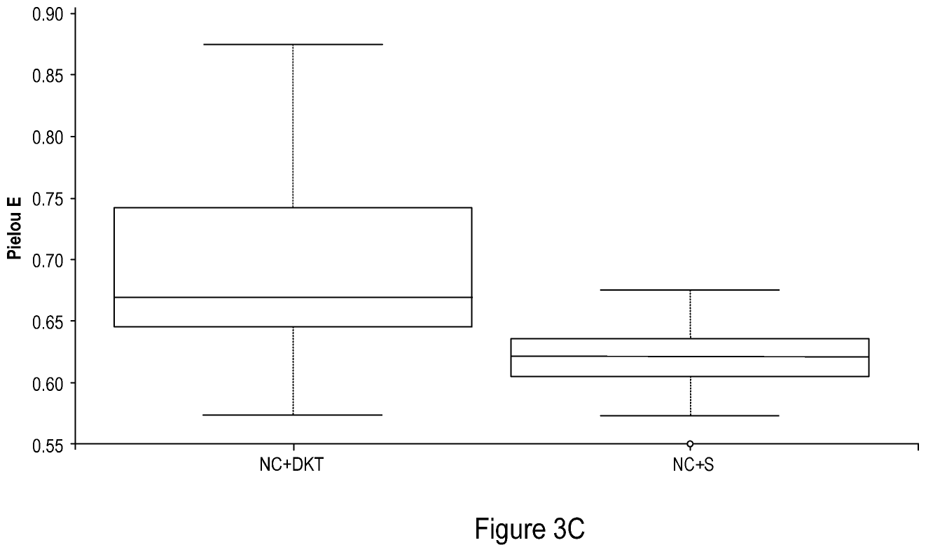

There was a significant difference in the constitution of the gut microbiome (p-value=0.036) in mice administered DKT and in mice administered vehicle, as shown in the unweighted Principal Coordinates Analysis (PCoA) of the microbiome (FIG. 3A).

The microbiomes of DKT and saline groups may be compared using alpha diversity metrics, such as Faith Phylogenetic Diversity (PD) and Pielou Evenness. Faith PD measures the phylogenetic diversity in the group, and Pielou Evenness measures the uniformity of the distribution of the various sequences found in a group. DKT-administered mice had significantly higher phylogenetic diversity (Faith PD P-Value=0.0043; FIG. 3B) and higher uniformity of distribution (Pielou Evenness P-Value=0.0058; FIG. 3C) than the saline-administered group.

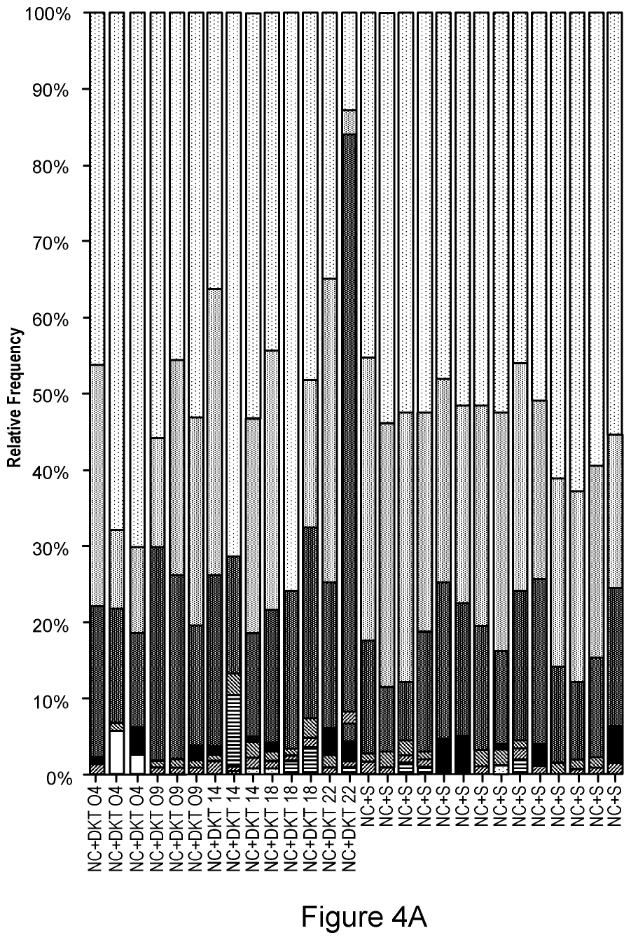

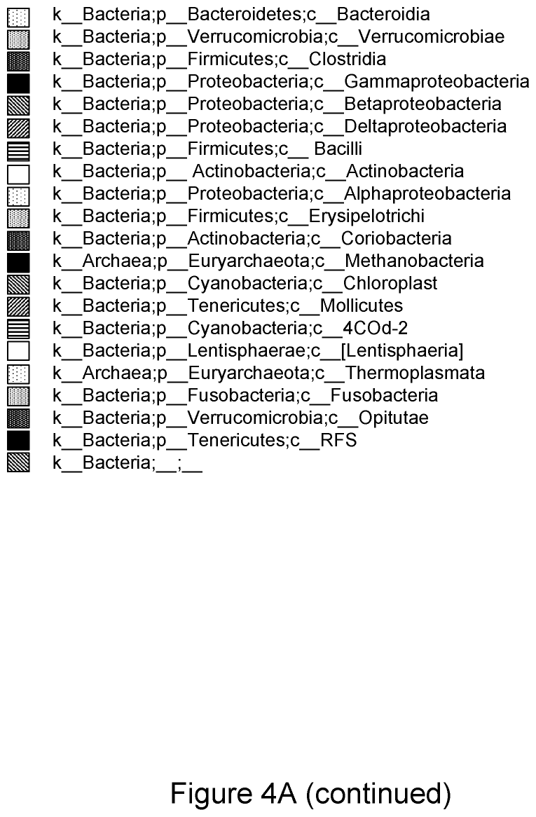

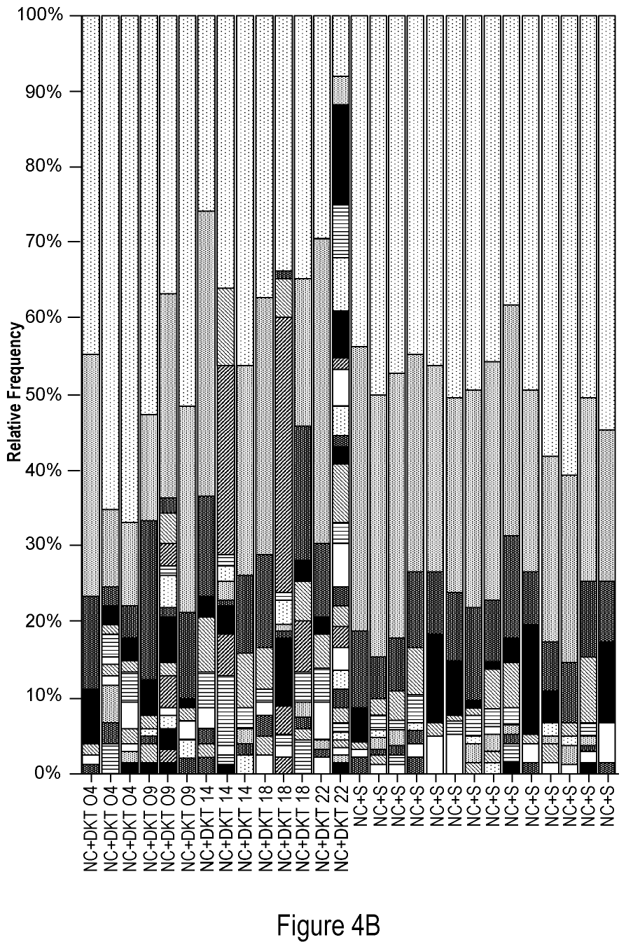

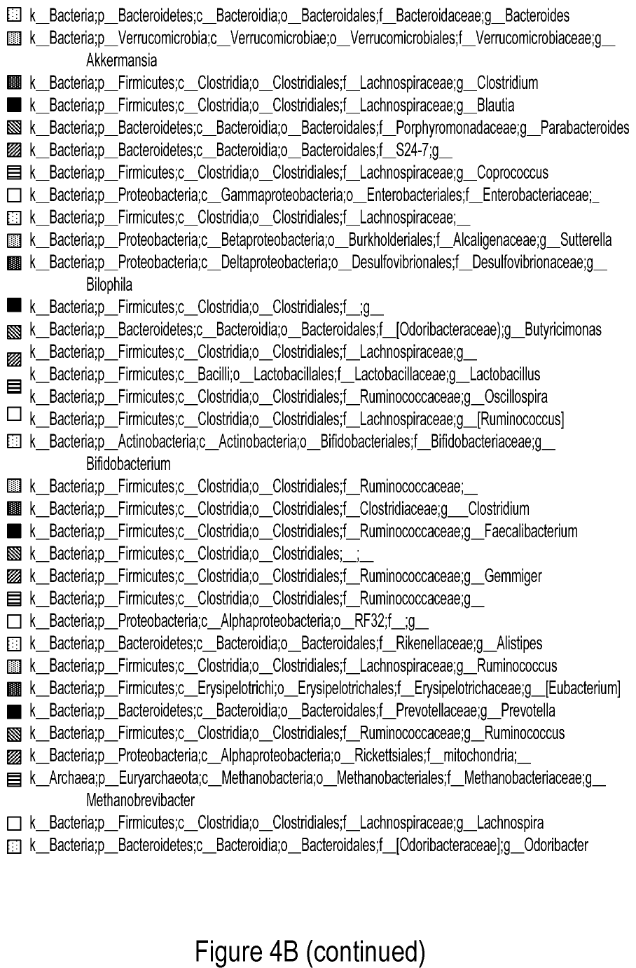

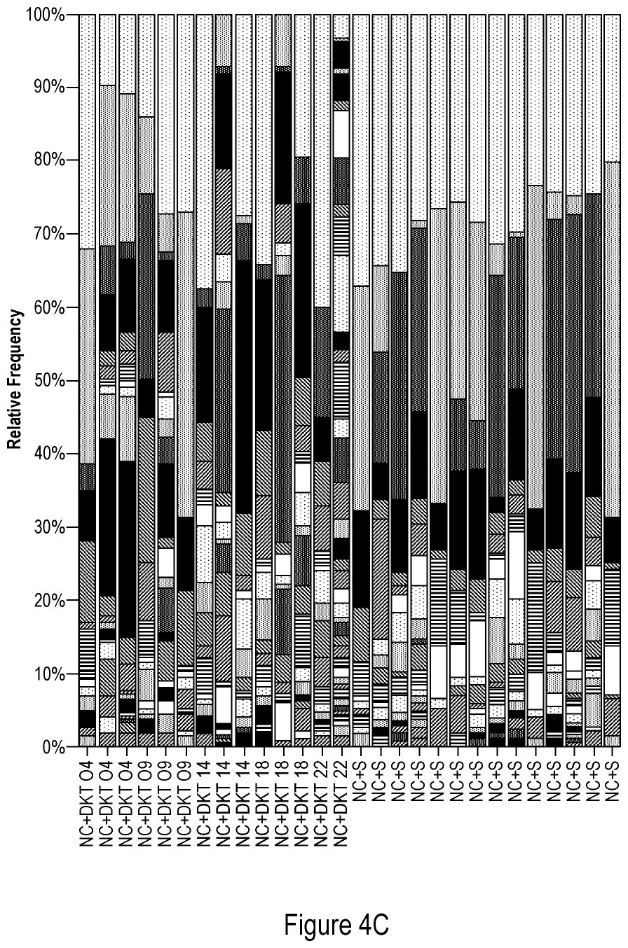

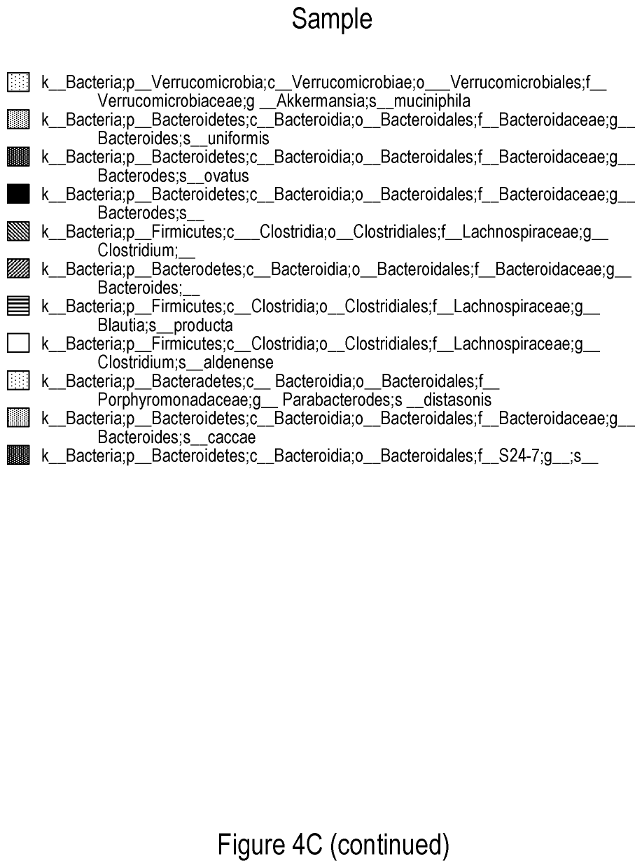

The variability in the microbiomes of individual subjects as well as treatment groups is presented in FIGS. 4A-4C. Each bar represents the microbiome of the fecal samples collected from a single cage (in which all mice were orally administered DKT or saline) on specific days (Days 4, 9, 14, 18, and 22). Each colour represents different taxa, depending on the taxa level; e.g. FIG. 4A categorizes the microbes by class (3 levels), FIG. 4B by genus (6 levels), and FIG. 4C by species (7 levels).

There were statistically significant differences (defined as P-Value<0.05 on statistical non-parametric Wilcoxon tests) in the levels of specific phyla, classes, and species between mice administered DKT and mice administered saline (Tables 1-3). Specifically, the phylum (Table 1) and class (Table 2) Verrucomicrobia was lower in DKT-administered mice compared to controls. In addition, the species Aerofaciens and Coprophilus, and the genus Parabacteroides are present at significantly higher levels in the microbiome of DKT-administered animals, compared to controls. Similarly, the orders of Clostridiales and Bacteroidales are present in the microbiome of DKT-administered mice at significantly higher levels compared to controls, while the Fragilis species are present at significantly lower levels (Table 3).

TABLE 1
The Phylum Verrucomicrobia is lower in mice administered
DKT compared to mice administered saline.

| Bacteria_Phylum_Level 2 | DKT | Saline |
|---|---|---|
| k_Bacteria; p_Verrucomicrobia | 4496 | 7783 |

TABLE 2
The Class Verrucomicrobiae is lower in mice administered
DKT compared to mice administered saline.

| Bacteria_Class_Level 3 | DKT | Saline |
|---|---|---|
| k_Bacteria; p_Verrucomicrobia; c_Verrucomicrobiae | 4496 | 7783 |

TABLE 3
Species present at significantly different levels in mice
administered DKT compared to mice administered saline.

| Bacteria_Species_Level 7 | DKT | Saline |
|---|---|---|
| k_Bacteria; p_Actlnobactoria; c_Coriobactcriia; o_Corloba cterialos; | 15 | 0 |
| f_Corlobactoriaccae; g_Collinsclla; s_acrofacicns | | |
| k_Bacteria; p_Bactcroidetes; c_Bacteroidia; o_Bacteroidales; f_Bacteroidaceae; | 914 | 0 |
| g_Bacteroides; s_coprophilus | | |
| k_Bacteria; p_Bactcroidetes; c_Bacteroidia; o_Bacteroidales; f_Bactcroidaceae; | 6 | 102 |
| g_Bacteroides; s_fragilis | | |
| k_Bacteria; p_Bactcroidetes; c_Bacteroidia; o_Bacteroidales; | 164 | 0 |
| f_Porphyromonadaceae; g_Parabacteroides; s_ | | |
| k_Bacteria; p_Bactcroidetes; c_Bacteroidia; o_Bacteroidales; f_S24-7; g_; s_ | 903 | 13 |
| k_Bacteria; p_Firmicutes; c_Clostridia; o_Clostridiales; _; _; _ | 66 | 0 |
| k_Bacteria; p_Firmicutes; c_Clostridia; o_Clostridiales; f_; g_; s_ | 355 | 16 |

### GC-MS Analysis:

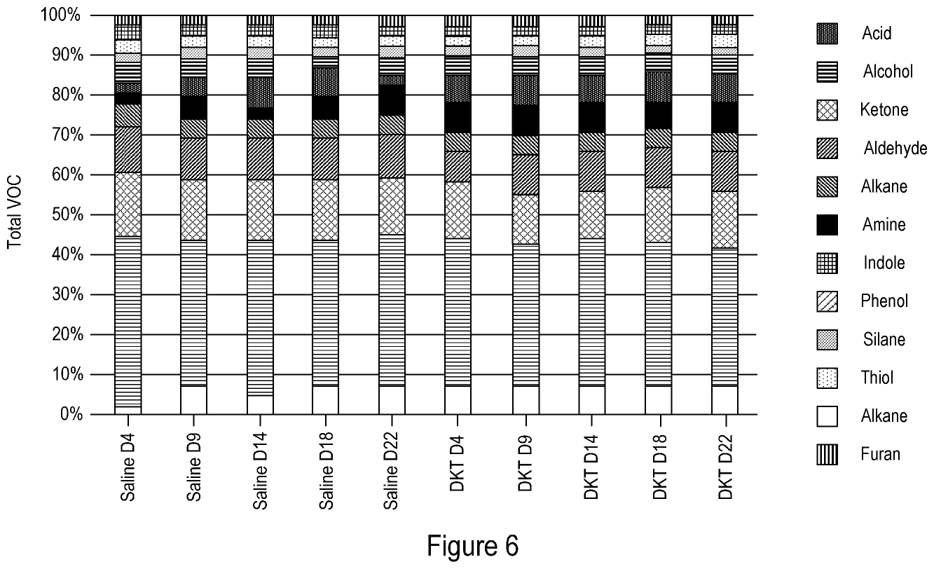

GC-MS was used to analyse feces samples to identify specific VOCs in the headspace of the samples. A total number of 433 VOCs were detected. The VOCs were from the families of acids, alcohols, ketones, aldehydes, alkanes, amine, indoles, phenols, silanes, thiols, alkenes and furanes. Excluding VOCs which appear in less than 50% of the samples resulted in total of 76 VOCs distributed to the same families. The percentage of acids, alcohols, ketones, aldehydes, alkanes, amine, indoles, phenols, silanes, thiols, alkenes and furanes in each one of the fecal samples is shown in FIG. 6.

Four compounds were significantly decreased in mice administered DKT: acetic acid; hydrazine, ethyl-ethanedioate (e.g. oxalic acid derivative); 1h-indole,5-methyl,2-phenyl; and 4-methyl,2-phenyl indole. The average area under curve (AUC) of these compounds was significantly lower in Wilcoxon test (P-Value<0.05) in mice administered DKT (all days) compared to mice administered saline.

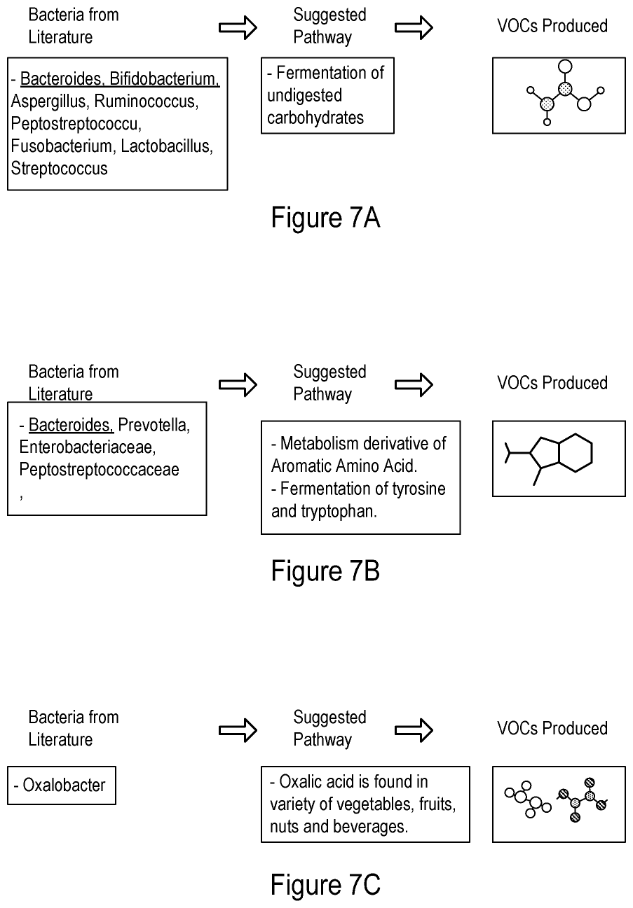

The formation of undigested carbohydrates is highly correlated with the activity of the gut microbiome. More specifically, certain phyla are associated with carbohydrate fermentation, among them are Bacteroides and Bifidobacterium (FIG. 7A). The data presented herein demonstrates that some Bacteroides, such as S. coprophilus, Parabacteroides and 524-7 are significantly increased in DKT compared to saline treatment groups while the Bacteroide S. fragilis is decreased (Table 3). Without being limited by a particular theory, an increase in Bacteroides (Table 3) may explain the decrease in acetic acid formation (previous paragraph and FIG. 6).

Bacteroides also play a role in the metabolism of aromatic amino acids, such as tyrosine and tryptophan (FIG. 7B). Without being limited by a particular theory, a decrease in some species of Bacteroides, such as S. fragilis, (see Table 3) in DKT mice may decrease production of aromatic amino acids and thereby decrease indole derivative levels (e.g., 1h-indole, 5-methyl, 2-phenyl and 4-methyl,2-phenyl indole) in the gut and feces of DKT-treated mice, as was found and described hereinabove.

Oxalic acid is found in many vegetables, fruits, and nuts and is thought be metabolized by Oxalobacter (FIG. 7C), and Oxalobacter levels decreased to near zero in DKT-treated mice (Table 3). Without being limited by a particular theory, lower Oxalobacter levels may have lead to the decrease in oxalic acid (e.g. hydrazine, ethyl-ethanedioate) that was observed and described hereinabove for DKT-treated mice.

### NanoScent Scent Recorder Analysis:

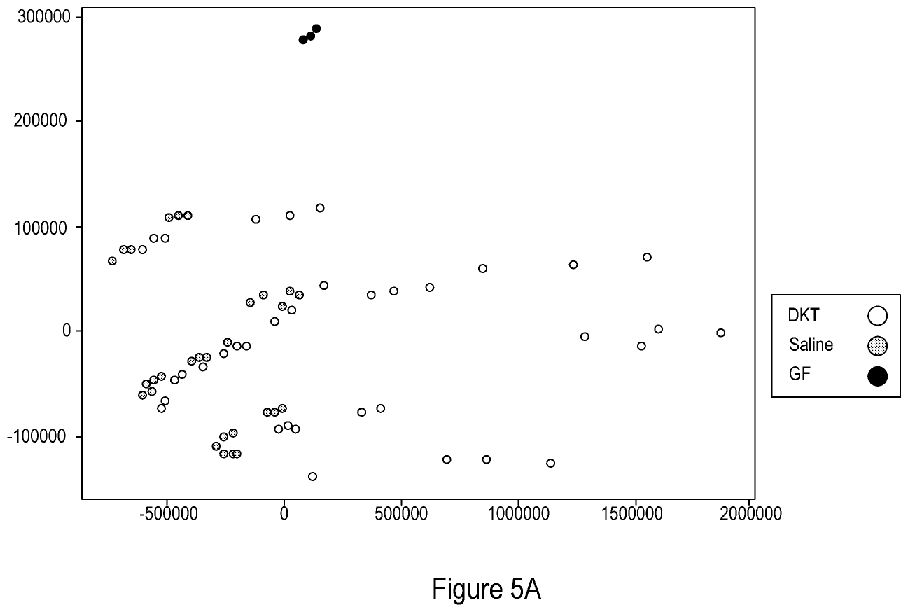

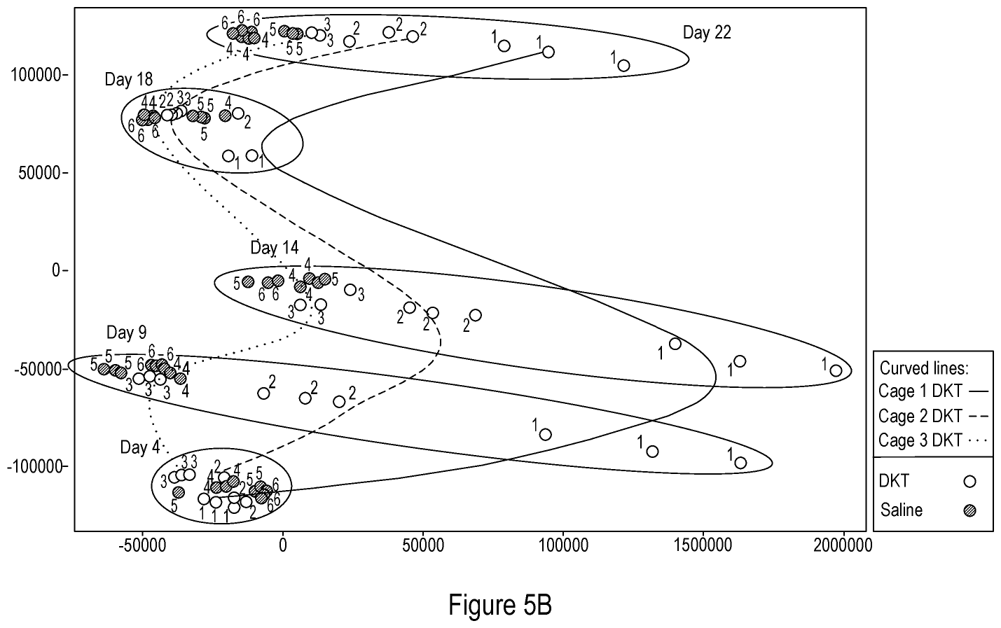

A graphical representation of the multiple signals obtained from the NanoScent Scent Recorder can be created using principal component analysis (PCA). The PCA map constructed based on NanoScent Scent Recorder signals for the comparison between the three groups: Germ Free (GF) mice, HMT mice administered DKT, and HMT mice administered saline are shown in FIG. 5A. The PCA demonstrates that feces collected from GF mice have a distinctive scent compared to HMT mice treated with either DKT or saline.

FIG. 5B shows the PCA map without the data from GF mice and indicates the day of sample collection for each sample (Day 4, Day 9, Day 14, Day 18, or Day 22). The PCA maps clearly demonstrate a separation between the feces scent of mice administered DKT compared to the feces scent of mice administered saline. Moreover, the feces scent from mice administered saline was more closely grouped compared to mice administered DKT, indicating the effect of DKT on the microbiome might differ slightly between subjects. While a significant difference between DKT administered mice and vehicle-administered mice was observed on Days 14 and 22 (FIG. 5B), there was no significant effect of DKT administration on Day 4. For the DKT-administered mice (indicated as numbers 1-3), the scent trend (illustrated as curved line between the samples) is similar, which indicates that the change in scent pattern between the first day of measurement until the last day is similar.

### Machine Learning Analysis:

The data obtained from the NanoScent Scent Recorder measurements for mice administered DKT and mice administered saline was divided into a training set (i.e. 70% of the digital input) and a test set (i.e. 30% of the digital input). Applying machine learning to the training set provided a pre-processed trained model, that can be used for inferring an unknown scent (i.e. scent recognition), i.e. capable of determining whether a mouse was administered DKT or saline. The accuracy for the comparison between GF mice, mice administered DKT, and mice administered saline was 90.5% on the test set (data not shown).

Conclusion: The results show that DKT administration alters the gut microbiome, the VOCs, and feces scent in mice. It is clear from the NanoScent Scent Recorder signals as well as from the GC-MS signals, that DKT ingestion alters the relative abundance of some VOCs (such as acetic acid; hydrazine, ethyl-ethanedioate; 1h-indole,5-methyl,2-phenyl; and 4-methyl,2-phenyl indole) in feces.

### Example 6

### Gut Microbiome, VOCs, and Fecal Scent Differ Based on Human Subjects' Gender, BMI, Physical Activity, or Diet

Goal: To evaluate the correlation between feces scent and the gut microbiome composition and VOCs. Specific goals were to determine which VOCs are shared by healthy human subjects and analyze which VOCs are characteristic of different groups of subjects.

Methods: Eleven healthy human subjects between the ages of 29-60 were recruited and gave their consent to participate in the study. Subjects filled in questionnaires at the beginning of the study, provided information regarding their general health and lifestyle and collected their feces five times within a period of two weeks. Subjects were trained in proper feces collection and storage via a) viewing a short video demonstrating the feces collection process; b) reading an instruction page, and c) discussing the protocol with the clinical trial manager. Subjects recorded the contents of the ingredients of their last meal prior to the fecal sample collection in a table. Feces samples were collected from all subjects, transferred to the laboratory for analysis under cool conditions, and kept at −20° C. until their analysis. Samples were analysed by 16S ribosomal DNA sequencing, GC-MS, and NanoScent Scent Recorder.

Among the 11 subjects, 5 were males and 6 females; 7 subjects had normal BMI, 3 were overweight and one was obese; seven subjects reported engaging in sports activity at least twice a week, and four reported not practicing any sports. Two subjects were vegetarian.

Results:

### Microbiome Analysis by 16S Ribosomal DNA Sequencing:

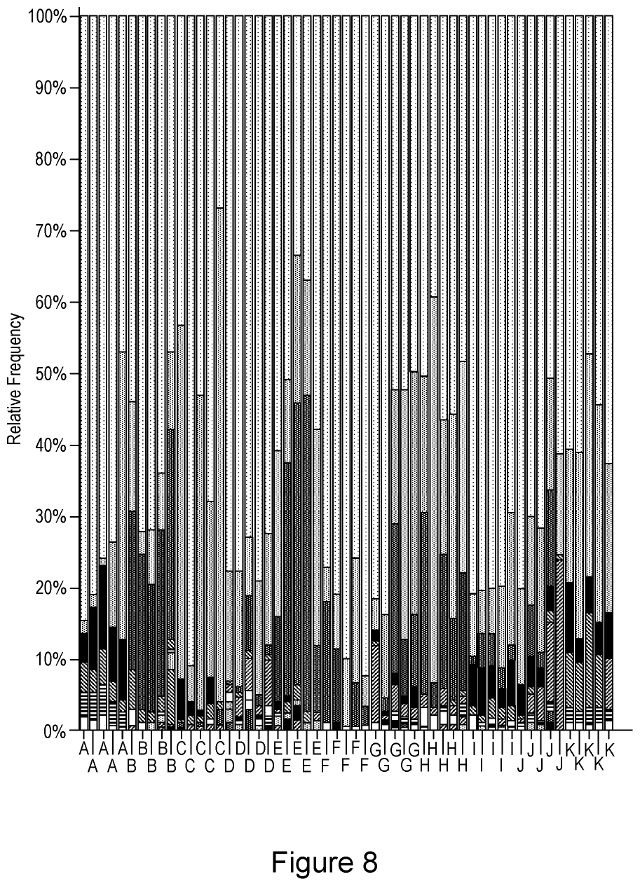

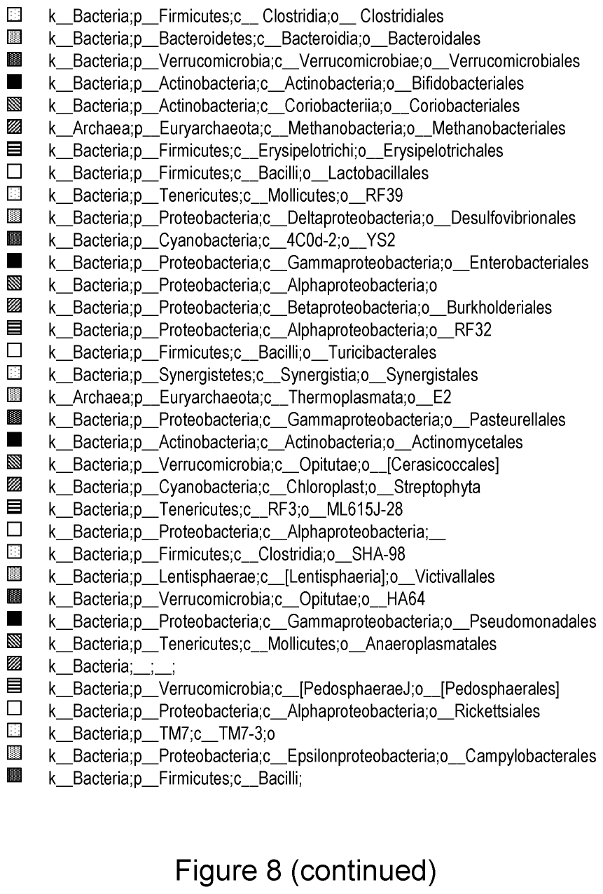

80% of the gut microbiome of healthy subjects comprises Firmicutes and Bacteroides (FIG. 8). This finding is consistent with previous reports in the literature, thereby affirming proper sample collection and processing methods. Samples collected from a single subject had a higher similarity of microbial DNA than samples collected from different subjects, indicating that the microbiome in an individual subject is relatively stable over time. The obese subject (Subject K) had lower diversity of the gut microbiome.

### GC-MS Analysis:

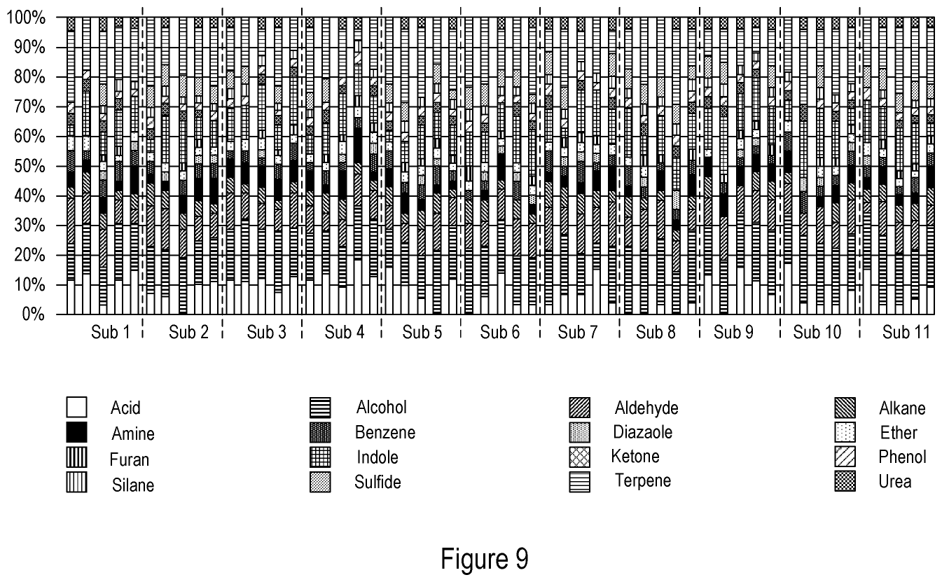

The VOCs detected in human samples were from the following VOC families: acid, alcohol, aldehyde, alkane, amine, benzene, diazole, ether, furan, indole, ketone, phenol, silane, sulfide, terpene, and, urea. Forty-six VOCs distributed among the VOC families were detected after excluding VOCs that appeared in less than 50% of the samples. The percentage of each VOC family in each one of the fecal samples is presented in FIG. 9. The data clearly demonstrate that VOCs, like microbial DNA, are similar in healthy subjects, and that there is a higher similarity between VOCs originating from feces collected from the same subject compared to VOCs from fecal samples of different subjects (i.e., lower intra-subject variability compared to inter-subject variability).

In order to test whether VOC distribution varied among different groups of subjects, subjects were divided into the following sub-groups:
Person ID: 11 healthy subjects
Gender: 6 Females, 5 Males
Age groups: 29-35, 35-45, 45-55, >55
BMI: 7—normal, 3—overweight, 1—obese
Sports: 7—Yes, 4—No
Vegetarian: 2—Yes, 9—No

The GC-MS dataset contains 11*5 (55) data points (number of subjects*number of samples), where each data point represents a sample having the following tags: name, gender, age, BMI, whether the person is engaged in sport activity (at least twice a week) and if the person eats a vegetarian only diet. Each data point in the GC/MS dataset is a list of VOCs found in the sample together with a number representing the amount of that molecule (i.e. the area under the curve).

There were significant differences in the expression of some VOCs based on the gender of the subject (Table 4), sports activity of the subject (Table 5), BMI of the subject (Table 6), and vegetarian diet of the subject (Table 7), as determined by GC-MS (P-Value<0.05 in Wilcoxon test).

Table 4 lists VOCs for which there was a statistically significant difference in VOC expression level between male and female subjects. Propanoic acid levels were significantly increased in the headspace of fecal samples from male subjects compared to female subjects, while 2-methyl butanal, Dihydroxy 2,5,8,11,14 . . . , 3-Methyl phenol, Dimethyl disulphide, and Dimethyl trisulfide in the headspace of fecal samples were significantly increased in female subjects.

TABLE 4
VOCs for which the VOC expression level
differs significantly based on gender

| Male | Female |
|---|---|
| Propanoic Acid | 2-methyl butanal |
| | dihydroxy-2,5,8,11,14- |
| | pentaoxacyclopentadecane |
| | 3-Methyl phenol |
| | Dimethyl disulfide |
| | Dimethyl trisulfide |

Table 5 lists VOCs for which there was a statistically significant difference in VOC expression level between subjects who reported practicing sports (at least twice a week) and subjects who reported not doing any sports activity. Benzene ethanol, 2-methyl butanal, 1-methyl 4-methyl (1-methylethyl) Benzene, 3-Methyl phenol, α-pinene, β-caryophyllene, and γ-terpinene levels were significantly increased in the headspace of fecal samples of non-active subjects compared to active subjects.

TABLE 5
VOCs for which the VOC expression level differs
significantly based on sport activity

| Active Subjects | Non-active Subjects |
|---|---|
| nd | Benzene ethanol |
| nd | 2-methyl butanal |
| nd | 1-methyl 4-methyl (1-methylethyl) Benzene |
| nd | 3-Methyl phenol |
| nd | α-pinene |
| nd | β-caryophyllene |
| nd | γ-terpinene |

nd—not detected

Table 6 lists VOCs for which there was a statistically significant difference in VOC expression level between subjects with different BMI indexes. Each column represents a different binary comparison. The group with the higher concentration of the VOC in the binary comparison is indicated in parentheses. 3-methyl 1-indole, 4-methyl Phenol, and δ-carene levels in the headspace of fecal samples were significantly increased in overweight subjects compared to subjects having normal BMI. In the comparison between obese subjects and subjects with normal BMI, Benzeneethanol, 4-methyl Phenol, Dimethyl disulphide, and Dimethyl trisulfide were significantly increased in the headspace of fecal samples from obese subjects, while Styrene was significantly increased in the headspace of fecal samples from normal subjects. In the comparison between obese subjects and overweight subjects, 3-methyl 1-indole, Dimethyl disulphide, and Dimethyl trisulfide were significantly increased in the headspace of fecal samples from obese subjects, while Styrene was significantly increased in the headspace of fecal samples from overweight subjects.

TABLE 6
VOCs for which the VOC expression level
differs significantly based on BMI

| Normal vs. | | |
|---|---|---|
| Overweight | Normal vs. Obese | Overweight vs. Obese |
| 3-methyl 1-indole | Benzeneethanol | Styrene (Over) |
| (Over) | (Obese) | |
| 4-methyl Phenol | Styrene (Normal) | 3-methyl 1-indole |
| (Over) | | (Obese) |
| δ-carene (Over) | 4-methyl Phenol | Dimethyl disulfide |
| | (Obese) | (Obese) |
| | Dimethyl disulfide | Dimethyl trisulfide |
| | (Obese) | (Obese) |
| | Dimethyl trisulfide | |
| | (Obese) | |

Table 7 contains a list of significant VOCs which were found to have P-value<0.05 in the comparison between vegetarian and non-vegetarian subjects. Benzene, 1-methyl, 4(1-methylethyl), 1-indole, Betacaryophyllene, β-pinene, δ-carene, and γ-terpinene levels were significantly increased in the headspace of fecal samples of non-vegetarian subjects compared to vegetarian subjects.

TABLE 7
VOCs for which the VOC expression level differs
significantly based on vegetarian diet

| Vegetarian | Non-vegetarian |
|---|---|
| nd | Benzene, 1-methyl, 4(1-methylethyl) |
| nd | 1-indole |
| nd | Betacaryophyllene |
| nd | β-pinene |
| nd | δ-carene |
| nd | γ-terpinene |

nd—not detected

### Machine Learning Analysis:

Machine learning algorithms for differentiating between the various groups were applied to the three datasets: 16S ribosomal DNA sequencing GC-MS, and NanoScent Scent Recorder. The GC-MS dataset included 46 VOCs that were detected after excluding VOCs that appeared in less than 50% of the samples. Table 8 shows the calculated accuracies of the different methods (i.e. NanoScent Scent Recorder, 16S ribosomal DNA sequencing and GC-MS) for predicting features based on sub-grouping.

TABLE 8
Calculated accuracies for predicting
features based on sub-grouping.

| Predicted | 16S ribosomal | | NanoScent Scent |
|---|---|---|---|
| Feature | DNA sequencing | GC-MS | Recorder |
| Person ID | 100% | 41% | 100% |
| Gender | 90.1% | 71% | 100% |
| Age Group | 100% | 59% | 79% |
| BMI | 90.1% | 64% | 93% |
| Sports | 100% | 59% | 88% |
| Vegetarian | 100% | 76% | 93% |

The prediction accuracy for identifying the gender, age group, BMI, active lifestyle, and vegetarian diet of a subject based on 16S ribosomal DNA sequencing was higher than 90% (Table 8, 2^{nd} column). Similarly, the prediction accuracy for identifying the gender, BMI, active lifestyle, and vegetarian diet of a subject based on NanoScent Scent Recorder was above 85% accuracy (Table 8, 1^{st} column). The prediction accuracy for identifying the gender, age group, BMI, active lifestyle, and vegetarian diet of a subject based on GC-MS signals (i.e. VOCs) were much lower (Table 8, 3^{rd} column), with 71% accuracy for differentiating between males and females and 76% for the comparison between vegetarian and non-vegetarian subjects. The remaining comparisons yielded accuracies lower than 65%.

Conclusion: In human subjects, the microbiome in fecal samples (as measured by 16S ribosomal DNA sequencing of human feces samples) had some intersubject variability and, to a lesser extent, intrasubject variability. Certain VOCs are present at significantly different levels in specific sub-groups (gender, age group, BMI, active lifestyle, and diet), as measured by GC-MS analysis of VOCs in headspace of human fecal samples. Surprisingly, the scent profile (measured by NanoScent Scent Recorder analysis in headspace of human fecal samples) had similar predictive accuracy of sub-group membership to 16S ribosomal DNA sequencing and was far better than GC-MS. These data demonstrate that NanoScent Scent Recorder measurements of human fecal samples may be used instead of GC-MS and/or 16S ribosomal DNA sequencing for VOC detection and/or microbiome evaluation.

Analyzing the microbiome of subjects based on the scent of bodily excretions provides a way for subjects to obtain relevant health and nutritional recommendations in real time, as illustrated in FIG. 1A. The starting microbiome profile of the subject and subsequent changes to the microbiome of the subject may be monitored periodically by measuring the scent of a biological sample, such as a stool sample. The subject may then receive feedback based on his/her microbiome profile. For example, the subject may be instructed to continue or discontinue taking a dietary supplement/pharmaceutical, or to increase or decrease the dosage.

In contrast, the analysis of VOCs by analytical methods such as SPME combined with GC-MS or SIFT-MS is complex, lengthy, costly and cannot be performed in real time.

The NanoScent Scent Recorder in the examples above comprises chemi-resistor sensors which change their resistivity or other electrical property in response to chemical stimulation caused by the presence and/or change in concentration of various chemo-signals. These chemi-resistors are based on nanomaterials, and thus can serve as a sensitive method for the detection of various scent agents without the need of special preparation or expert analysis. Each sensor (or sensing element) can respond to different VOCs, and each VOC can be absorbed on different sensing element. Since these sensors may be cross-reactive, they therefore may be capable of responding to a variety of VOCs, providing a pattern or signature rather than specific identification.

Here, we suggest a user-friendly method and apparatus for regularly monitoring the gaseous phase of biological samples and providing health and nutritional recommendations based on machine learning of the pattern of sensor signals from the scent reader. This allows continuous scent detection without the need for sample preparation and storage. Additionally, it enables continuous monitoring of metabolic, diet and general health parameters in a rapid and inexpensive manner.

1. (canceled)

2. A method of detecting changes in the microbiome profile of a subject comprising the steps of:
a. profiling a microbiome of said subject based on the gaseous phase of a first sample obtained from said subject at a first timepoint;
b. profiling the microbiome of said subject based on the gaseous phase of a second sample obtained from said subject at a second timepoint; and
c. comparing the microbiome profile of said subject at said first timepoint to the microbiome profile of said subject at said second timepoint, wherein if the microbiome profiles at the two timepoints are different, then a change in the microbiome profile of said subject is detected,
wherein profiling the microbiome of the subject from the first sample and/or the second sample obtained from said subject comprises:
a. exposing the gaseous phase of said sample to a scent recorder comprising one or more sensors;
b. receiving a pattern of sensor signals from said scent recorder;
c. providing said pattern of sensor signals to a model trained to associate said pattern of sensor signals with a microbiome profile; and
d. determining the microbiome profile of said subject based on said association,
and wherein said sent recorder comprises one or more chemi-resistors comprise metallic nanoparticles coated with an organic ligand shell.

3. The method of claim 2, wherein the subject's intake of Daikenchuto (DKT) was altered between said first and second timepoints.

4. A method of training a model to associate the microbiome profiles of subjects with patterns of sensor signals from a scent recorder, the method comprising the steps of:
a. providing one or more samples obtained from one of said subjects;
b. exposing the gaseous phase of said one or more samples to a scent recorder;
c. receiving a pattern of sensor signals from said scent recorder;
d. identifying one or more microbes in said sample using molecular techniques;
e. correlating said pattern of sensor signals with said one or more microbes identified; and
f. repeating steps (a) through (e) with one or more samples from one or more additional subjects, to train the model to associate patterns of sensor signals from said scent recorder with said one or more microbes identified,
wherein the sent recorder comprises one or more chemi-resistors comprise metallic nanoparticles coated with an organic ligand shell.

5. The method of claim 4, wherein said molecular techniques comprise multiplex real-time polymerase chain reaction (PCR), RFLP of the 16S rRNA, amplified rDNA restriction analysis (ARDRA), pyro sequencing, whole genome sequencing, Fluorescent In Situ Hybridization (FISH), shotgun analysis, denaturing or temperature gradient gel electrophoresis (DGGE/TGGE), or a combination thereof.

6. A method of recommending/prescribing a diet to a subject comprising the steps of:
a. profiling a microbiome of said subject based on a sample obtained from said subject;
b. diagnosing said subject with a dietary condition based on the microbiome profile; and
c. making a first dietary recommendation to said subject based on said microbiome profile and said dietary condition,
wherein profiling the microbiome of the subject from the first sample and/or the second sample obtained from said subject comprises:
a. exposing the gaseous phase of said sample to a scent recorder comprising one or more sensors;
b. receiving a pattern of sensor signals from said scent recorder;
c. providing said pattern of sensor signals to a model trained to associate said pattern of sensor signals with a microbiome profile; and
d. determining the microbiome profile of said subject based on said association,
and wherein said sent recorder comprises one or more chemi-resistors comprise metallic nanoparticles coated with an organic ligand shell.

7. The method of claim 6, wherein said dietary recommendation comprises ingesting one or more food supplements, one or more probiotics, one or more prebiotic foods, one or more functional foods, one or more enriched foods, or a combination thereof.

8. The method of claim 7, wherein said functional food comprises Daikenchuto (DKT).

9. A method of claim 7, further comprising the steps of:
a. recommending/prescribing a diet to a subject;
b. obtaining a second sample from said subject at a second timepoint, wherein said second timepoint is a set time after the implementation of said dietary recommendation by said subject;
c. profiling the microbiome of said subject based on said second sample obtained from said subject; and
d. diagnosing said subject with a dietary condition based on the presence, absence, or relative abundance of at least one microbe in said microbiome profile;
wherein
i. if the presence, absence, or relative abundance of said at least one microbe in said microbiome profile of said subject at the second timepoint is at the desired level compared to the level at the first timepoint, then said first dietary recommendation is discontinued and the microbiome of said subject is monitored regularly;
ii. if the presence, absence, or relative abundance of said at least one microbe in said microbiome profile of said subject at the second timepoint is closer to the desired level compared to the level at the first timepoint, then said first dietary recommendation is continued and the microbiome of said subject is monitored regularly until condition (i) is met; and
iii. if the presence, absence, or relative abundance of at least one microbe in said microbiome profile of said subject at the second timepoint is farther from the desired level compared to the level at the first timepoint, then said subject is recommended a second dietary recommendation.

10.-15. (canceled)

16. The method of claim 2, wherein said microbiome comprises an intestinal microbiome, a stomach microbiome, a gut microbiome, an oral microbiome, or a combination thereof.

17. (canceled)

18. The method of claim 2, wherein said one or more sensors are configured to detect one or more volatile organic compounds (VOCs) from the gaseous phase of said biological sample.

19. The method of claim 18, wherein said one or more VOCs comprises 1-methyl 4-methyl (1-methylethyl) Benzene, 2-methyl butanal, 3-methyl 1-indole, 3-Methyl phenol, 4-methyl Phenol, Benzene ethanol, Dihydroxy 2,5,8,11,14, Dimethyl disulfide, Dimethyl trisulfide, acetic acid, Propanoic Acid, Styrene, a-pinene, b-caryophyllene, g-terpinene, d-carene, hydrazine, ethyl-ethanedioate; lh-indole, 5-methyl, 2-phenyl; 4-methyl, 2-phenyl indole, or a combination thereof.

20. The method of claim 2, wherein said subject comprises a human subject.

21. The method of claim 2, wherein said sample comprises a biological sample.

22. The method of claim 21, wherein said biological sample comprises a stool, breath, urine, skin, saliva, sweat, or blood sample.

23. The method of claim 4, wherein said microbiome comprises an intestinal microbiome, a stomach microbiome, a gut microbiome, an oral microbiome, or a combination thereof.

24. The method of claim 4, wherein said one or more sensors are configured to detect one or more volatile organic compounds (VOCs) from the gaseous phase of said biological sample.

25. The method of claim 24, wherein said one or more VOCs comprises 1-methyl 4-methyl (1-methylethyl) Benzene, 2-methyl butanal, 3-methyl 1-indole, 3-Methyl phenol, 4-methyl Phenol, Benzene ethanol, Dihydroxy 2,5,8,11,14, Dimethyl disulfide, Dimethyl trisulfide, acetic acid, Propanoic Acid, Styrene, a-pinene, b-caryophyllene, g-terpinene, d-carene, hydrazine, ethyl-ethanedioate; lh-indole, 5-methyl, 2-phenyl; 4-methyl, 2-phenyl indole, or a combination thereof.

26. The method of claim 4, wherein said subject comprises a human subject.

27. The method of claim 4, wherein said sample comprises a biological sample.

28. The method of claim 27, wherein said biological sample comprises a stool, breath, urine, skin, saliva, sweat, or blood sample.
